(19) United States
(12) Patent Application Publication
(10) Pub. No.: US 2005/0113294 A1
(43) Pub. Date: May 26, 2005

(54) Carboxamide and amino derivatives and methods of their use

(75) Inventor: Roland E. Dolle, King of Prussia, PA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Adolor Corporation.

(21) Appl. No.: 10/719,627

(22) Filed: Nov. 21, 2003

(51) Int. Cl. A61K 38/12; A61K 31/4709; C07K 5/12; C07D 43/02

(52) U.S. Cl. 514/9; 514/310; 530/317; 546/146

(57) ABSTRACT

Carboxamide and amino derivatives, pharmaceutical compositions containing these compounds, and methods for their pharmaceutical use are disclosed. In certain embodiments, the carboxamide derivatives are ligands of the δ opioid receptor and are useful, inter alia, for treating and/or preventing pain, anxiety, gastrointestinal disorders, and other δ opioid receptor-mediated conditions.

### FIELD OF THE INVENTION

The invention relates to carboxamide and amino derivatives, pharmaceutical compositions containing these compounds, and methods for their pharmaceutical use. In certain embodiments, the carboxamide and amino derivatives are ligands of the δ opioid receptor and are useful, inter alia, for treating and/or preventing pain, anxiety, gastrointestinal disorders, and other δ opioid receptor-mediated conditions.

### BACKGROUND OF THE INVENTION

There are at least three different opioid receptors (μ, δ, and κ) that are present in both central and peripheral nervous systems of many species, including humans. Lord, J. A. H., et al., Nature, 1977, 267, 495. Activation of the δ opioid receptors induces analgesia in various animal models. Moulin, et al., Pain, 1985, 23, 213. Some work suggests that the analgesics working at δ opioid receptors do not have the attendant side effects associated with μ and κ opioid receptor activation. Galligan, et al., J. Pharm. Exp. Ther., 1985, 229, 641. The δ opioid receptor has also been identified as having a role in circulatory systems. Ligands for the δ receptor have also been shown to possess immunomodulatory activities. Dondio, et al., Exp. Opin. Ther. Patents, 1997, 10, 1075. Further, selective δ opioid receptor agonists have been shown to promote organ and cell survival. Su, T-P, Journal of Biomedical Science, 2000, 9(3), 195-199. Ligands for the 6 opioid receptor may therefore find potential use as analgesics, as antihypertensive agents, and/or as immunomodulatory agents.

Numerous selective δ opioid ligands are peptidic in nature and thus are unsuitable for administration by systemic routes. Several non-peptidic δ opioid receptor ligands have been developed. See, for example, E. J. Bilsky, et al., Journal of Pharmacology and Experimental Therapeutics, 1995, 273(1), 359-366; WO 93/15062, WO 95/04734, WO 95/31464, WO 96/22276, WO 97/10216, WO 01/46192, WO 02/094794, WO 02/094810, WO 02/094811, WO 02/094812, WO 02/48122, WO 03/029215, WO 03/033486, JP-4275288, EP-A-0,864,559, U.S. Pat. No. 5,354,863, U.S. Pat. No. 6,200,978, U.S. Pat. No. 6,436,959 and U.S. 2003/0069241.

While there are a large number of non-peptidic δ opioid receptor modulators, there is still an unfulfilled need for compounds with selective δ opioid receptor activity that may be used in methods to provide beneficial pharmaceutical characteristics while minimizing undesirable side effects. The present invention is directed to these, as well as other important ends.

### SUMMARY OF THE INVENTION

The invention is generally related to carboxamide and amino derivatives, pharmaceutical compositions containing these compounds, methods for their pharmaceutical use, and radiolabeled and isotopically labeled derivatives of the carboxamide and amino derivatives.

In one embodiment, the invention is directed to compounds of formula I: 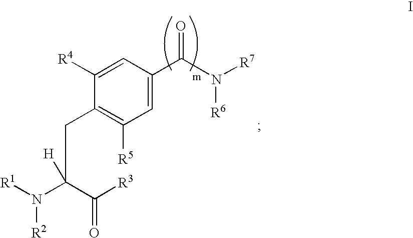
or a stereoisomer, prodrug, pharmaceutically acceptable salt, hydrate, solvate, acid salt hydrate, N-oxide or isomorphic crystalline form thereof.
wherein:
R¹ and R² are each, independently, H, alkyl, alkenyl, or —C(═NH)NH₂, provided that at least one of R¹ and R² is other than —C(═NH)NH₂, or R¹ and R² taken together with the nitrogen atom to which they are attached form a 4- to 8-membered heterocycloalkyl ring wherein the heterocycloalkyl ring is optionally interrupted with one additional O, N, or S heteroatom;
R³ is -[J]ₒ-X, 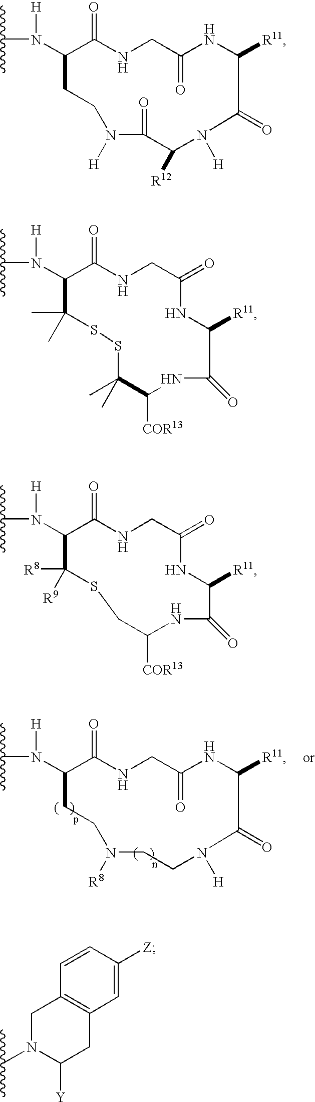
J is [NR^{8a}(C(R^{9a})(R¹⁰))ₙ—C(═O)];
X is OR⁸, NR^{8b}R^{9b}, or —N(R^{8c})CH(R^{9c})(R¹⁰);
R⁴ and R⁵ are each, independently, H or methyl;
R⁶ and R⁷ are each, independently, H or alkyl, or R⁶ and R⁷ taken together with the nitrogen atom to which they are attached form a 4- to 8-membered heterocycloalkyl ring wherein the heterocycloalkyl ring is optionally interrupted with one additional O, N, or S heteroatom;
R⁸ and R⁹ are each, independently, H or alkyl;
R^{8a} and R^{9a} are each, independently, H or alkyl, or R^{8a} and R^{9a} taken together with the nitrogen and carbon atoms through which they are connected form a 4- to 14-membered heterocycloalkyl ring wherein the heterocycloalkyl ring is optionally interrupted with one additional O, N, or S heteroatom;
R^{8b} and R^{9b} are each, independently, H or alkyl, or R^{8b} and R^{9b} taken together with the nitrogen atom to which they are attached form a 4- to 8-membered heterocycloalkyl ring wherein the heterocycloalkyl ring is optionally interrupted with one additional O, N, or S heteroatom;
R^{8c} and R^{9c} are each, independently, H or alkyl, or R^{8c} and R^{9c} taken together with the nitrogen and carbon atoms through which they are connected form a 4- to 8-membered heterocycloalkyl ring, wherein the heterocycloalkyl ring is optionally interrupted with one additional O, N, or S heteroatom;
R¹⁰ is —(C(R¹⁴)(R¹⁵))_{q}—R¹⁶;
R¹¹ and R¹² are each, independently, H or —C(R¹⁴)(R¹⁵)R¹⁰;
R¹³ is —OR¹⁴ or —NR¹⁴R¹⁵;
R¹⁴ and R¹⁵ are each, independently, H or alkyl;
R¹⁶ is H, alkyl, cycloalkyl, heterocycloalkyl, aryl, heteroaryl, —CR¹⁴R¹⁵OH, —CR¹⁴R¹⁵S(O)ᵣR⁹, —CR¹⁴R¹⁵COOR⁴, —CR¹⁴R¹⁵CONHR⁴, —CR¹⁴R⁵NHR⁴, or —CR¹⁴R¹⁵NH(C═NH)NH₂;
Y is heteroaryl;
Z is H or —OR⁸;
m is the integer 0 or 1;
n is the integer from 1 to 4;
o is an integer from 0 to 16;
p is the integer from 0 to 3;
q is an integer from 0 to 6; and
r is the integer 0 to 2.

In another aspect, the invention is directed to pharmaceutical compositions, comprising:
a pharmaceutically acceptable carrier; and
an effective amount of a carboxamide or amino derivative of formula I. In certain embodiments, the pharmaceutical composition further comprises an effective amount of at least one opioid.

In yet another aspect, the invention is directed to methods of binding opioid receptors, preferably δ opioid receptors, in a patient in need thereof, comprising the step of:
administering to said patient an effective amount of a carboxamide or amino derivative of formula I.

In preferred embodiments, the binding modulates the activity of the receptor. In certain other preferred embodiments, the binding agonizes the activity of said opioid receptors.

In other aspects, the invention is directed to methods of preventing or treating pain, comprising the step of:
administering to a patient in need thereof an effective amount of a carboxamide or amino derivative of formula I.

In another aspect, the invention is directed to methods for preventing or treating gastrointestinal dysfunction, comprising the step of:
administering to a patient in need of such treatment an effective amount of a carboxamide or amino derivative of formula I.

In another aspect, the invention is directed to methods for preventing or treating ileus, comprising the step of:
administering to a patient in need of such treatment an effective amount of a carboxamide or amino derivative of formula I.

In another aspect, the invention is directed to methods for preventing or treating a urogenital tract disorder, comprising the step of:
administering to a patient in need of such treatment an effective amount of a carboxamide or amino derivative of formula I.

In another aspect, the invention is directed to methods of preventing or treating an immunomodulatory disorder, comprising the step of:
administering to a patient in need thereof an effective amount of a carboxamide or amino derivative of formula I.

In another aspect, the invention is directed to methods of preventing or treating an inflammatory disorder, comprising the step of:
administering to a patient in need thereof an effective amount of a carboxamide or amino derivative of formula I.

In another aspect, the invention is directed to methods of preventing or treating a respiratory function disorder, comprising the step of:
administering to a patient in need thereof an effective amount of a carboxamide or amino derivative of formula I.

In another aspect, the invention is directed to methods for preventing or treating anxiety, comprising the step of:
administering to a patient in need of such treatment an effective amount of a carboxamide or amino derivative of formula I.

In another aspect, the invention is directed to methods for preventing or treating a mood disorder, comprising the step of:
administering to a patient in need of such treatment an effective amount of a carboxamide or amino derivative of formula I.

In another aspect, the invention is directed to methods for preventing or treating a stress-related disorder, comprising the step of:
administering to a patient in need of such treatment an effective amount of a carboxamide or amino derivative of formula I.

In another aspect, the invention is directed to methods for preventing or treating sympathetic nervous system disorder, comprising the step of:
administering to a patient in need of such treatment an effective amount of a carboxamide or amino derivative of formula I.

In another aspect, the invention is directed to methods for preventing or treating tussis, comprising the step of:
administering to a patient in need of such treatment an effective amount of a carboxamide or amino derivative of formula I.

In another aspect, the invention is directed to methods for preventing or treating a motor disorder, comprising the step of:
administering to a patient in need of such treatment an effective amount of a carboxamide or amino derivative of formula I.

In another aspect, the invention is directed to methods for treating a traumatic injury to the central nervous system, comprising the step of:
administering to a patient in need of such treatment an effective amount of a carboxamide or amino derivative of formula I.

In another aspect, the invention is directed to methods for preventing or treating stroke, comprising the step of:
administering to a patient in need of such treatment an effective amount of a carboxamide or amino derivative of formula I.

In another aspect, the invention is directed to methods for providing cardioprotection following myocardial infarction, comprising the step of:
administering to a patient in need of such treatment an effective amount of a carboxamide or amino derivative of formula I.

In another aspect, the invention is directed to methods for treating a condition selected from the group consisting of shock, brain edema, cerebral ischemia, cerebral deficits subsequent to cardiac bypass surgery and grafting, systemic lupus erythematosus, Hodgkin's disease, Sjogren's disease, epilepsy, and rejection in organ transplants and skin grafts, comprising the step of:
administering to a patient in need of such treatment an effective amount of a carboxamide or amino derivative of formula I.

In another aspect, the invention is directed to methods for treating substance addiction, comprising the step of:
administering to a patient in need of such treatment an effective amount of a carboxamide or amino derivative of formula I.

In another aspect, the invention is directed to methods of producing or maintaining an anesthetic state, comprising the step of:
administering to a patient in need of such treatment an effective amount of a carboxamide or amino derivative of formula I.

Preferably, the carboxamide or amino derivative of formula I is co-administered with an anesthetic agent selected from the group consisting of an inhaled anesthetic, a hypnotic, an anxiolytic, a neuromuscular blocker and an opioid.

In certain aspects, the invention is directed to the radiolabeled derivatives and the isotopically labeled derivatives of carboxamide or amino derivative of formula I.

### DETAILED DESCRIPTION OF ILLUSTRATIVE EMBODIMENTS

The invention relates to carboxamide and amino derivatives, pharmaceutical compositions containing these compounds, and methods for their pharmaceutical use. In certain embodiments, the carboxamide and amino derivatives are ligands of the δ opioid receptor and are useful, inter alia, in methods for treating and/or preventing diseases and conditions mediated or modulated by the δ opioid receptor, including, pain, gastrointestinal disorders, ileus, urogenital tract disorders, immunomodulatory disorders, inflammatory disorders, respiratory function disorders, anxiety, mood disorders, stress-related disorders, attention deficit hyperactivity disorder, sympathetic nervous system disorder, tussis, motor disorder, traumatic injury, stroke, cardiac arrhythmia, glaucoma, sexual dysfunction, shock, brain edema, cerebral ischemia, cerebral deficits subsequent to cardiac bypass surgery and grafting, systemic lupus erythematosus, Hodgkin's disease, Sjogren's disease, epilepsy, rejection in organ transplants and skin grafts, and substance addiction. In certain other embodiments, the carboxamide and amino derivatives are ligands of the δ opioid receptor and are useful, inter alia, in methods for improving organ and cell survival, methods for providing cardioprotection following myocardial infarction, in methods for reducing the need for anesthesia, in methods for providing and maintaining an anesthetic state, and in methods of detecting, imaging or monitoring degeneration or dysfunction of opioid receptors in a patient.

As employed above and throughout the disclosure, the following terms, unless otherwise indicated, shall be understood to have the following meanings.

“Alkyl” refers to an optionally substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms, herein referred to as “lower alkyl,” being preferred. Alkyl groups include, but are not limited to, methyl, ethyl, n-propyl, isopropyl, n-butyl, isobutyl, sec-butyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

“Amino acid” refers to an optionally further substituted alkyl-CO₂H having a primary secondary or tertiary amino substituent attached to a carbon atom within the alkyl chain, wherein alkyl is as herein defined. In some embodiments, the secondary or tertiary amino substituent is attached to the alkyl chain at two carbon atoms of the chain, thereby forming a heterocycloalkyl ring, such as in the non-limiting example of proline. In some embodiments the amino acids may be chiral. In other embodiments they may be naturally occurring. In still other embodiments, the amino acids may be homologues of naturally occurring amino acids in racemic form or in any of the individually possible chiral forms, or mixtures thereof.

“Cycloalkyl” refers to an optionally substituted, alkyl group having one or more rings in their structures having from about 3 to about 20 carbon atoms (and all combinations and subcombinations of ranges and specific numbers of carbon atoms therein), with from about 3 to about 10 carbon atoms being preferred. Multi-ring structures may be bridged or fused ring structures. Groups include, but are not limited to, cyclopropyl, cyclobutyl, cyclopentyl, cyclohexyl, cyclooctyl, 2-[4-isopropyl-1-methyl-7-oxa-bicyclo[2.2.1]heptanyl], 2-[1,2,3,4-tetrahydro-naphthalenyl], and adamantyl.

“Alkylcycloalkyl” refers to an optionally substituted ring system comprising a cycloalkyl group having one or more alkyl substituents, wherein cycloalkyl and alkyl are each as previously defined. Exemplary alkylcycloalkyl groups include 2-methylcyclohexyl, 3,3-dimethylcyclopentyl, trans-2,3-dimethylcyclooctyl, and 4-methyldecahydronaphthalenyl.

“Heterocycloalkyl” refers to an optionally substituted, mono-, di-, tri-, or other multicyclic aliphatic ring system that includes at least one, and preferably from 1 to about 4 sulfur, oxygen, or nitrogen heteroatom ring members. Heterocycloalkyl groups can have from about 3 to about 20 carbon atoms (and all combinations and subcombinations of ranges and specific numbers of carbon atoms therein), with from about 4 to about 10 carbons being preferred. In other preferred embodiments, the heterocycloalkyl groups have from about 4 to about 8 ring members, wherein 1 or 2 members are sulfur, oxygen, or nitrogen and the remaining members are carbon atoms. The heterocycloalkyl group may be unsaturated, and may also be fused to aromatic rings. Examples of heterocycloalkyl groups include, for example, tetrahydrofuranyl, tetrahydrothienyl, piperidinyl, pyrrolidinyl, isoxazolidinyl, isothiazolidinyl, pyrazolidinyl, oxazolidinyl, thiazolidinyl, piperazinyl, morpholinyl, piperadinyl, decahydroquinolyl, octahydrochromenyl, octahydro-cyclopenta[c]pyranyl, 1,2,3,4,-tetrahydroquinolyl, octahydro-[2]pyrindinyl, decahydro-cycloocta[c]furanyl, and imidazolidinyl.

In embodiments in which a heterocycloalkyl ring is formed, for example, by R^{8a} and R^{9a}, R^{8c} and R^{9d}, R^{8e} and R^{9e}, or R^{9e} and R^{9e}, together with the nitrogen and carbon atoms through which they are connected, R¹ and R², R⁶ and R or R^{8b} and R^{9b} together with the nitrogen atom to which they are attached, there are desirably at least two ring carbons in either direction along the ring separating the nitrogen atom of the heterocycloalkyl ring and an optional, additional heteroatom interrupting the ring. Thus, for example, if a heterocycloalkyl ring is formed by taking together R¹ and R², R⁶ and R⁷, R^{8a} and R^{9a}, R^{8c} and R^{9c}, R^{8d} and R^{9d}, or R^{8e} and R^{9e}, and an additional optional heteroatom such as oxygen, the resulting heterocycloalkyl ring may be: 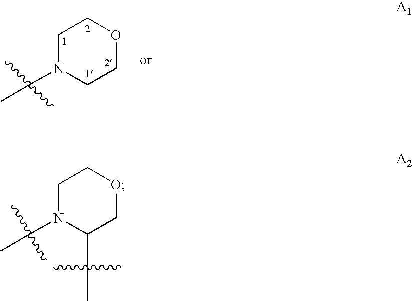
but not:

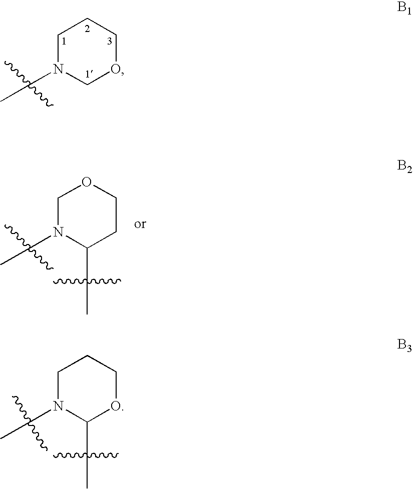

In examples A₁ and A₂, there are two ring carbons in either direction separating the nitrogen atom from the interrupting oxygen atom. In examples B₁, B₂ and B₃, while there are three ring carbon atoms separating the nitrogen from the interrupting oxygen atom when proceeding around the ring in a first direction, there is only one ring carbon atom separating the nitrogen and oxygen atoms when proceeding around the ring in the alternative direction.

“Alkylheterocycloalkyl” refers to an optionally substituted ring system comprising a heterocycloalkyl group having one or more alkyl substituents, wherein heterocycloalkyl and alkyl are each as previously defined. Exemplary alkylheterocycloalkyl groups include 2-methylpiperidinyl, 3,3-dimethylpyrrolidinyl, trans-2,3-dimethylmorpholinyl, and 4-methyldecahydroquinolinyl.

“Alkenyl” refers to an alkyl group having from about 2 to about 10 carbon atoms and one or more double bonds (and all combinations and subcombinations of ranges and specific numbers of carbon atoms therein), wherein alkyl is as previously defined. Alkenyl groups can be optionally substituted.

“Alkynyl” refers to an alkyl group having from about 2 to about 10 carbon atoms and one or more triple bonds (and all combinations and subcombinations of ranges and specific numbers of carbon atoms therein), wherein alkyl is as previously defined. Alkynyl groups can be optionally substituted.

“Aryl” refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

“Aralkyl” refers to alkyl radicals bearing an aryl substituent and have from about 6 to about 50 carbon atoms (and all combinations and subcombinations of ranges and specific numbers of carbon atoms therein), with from about 6 to about 10 carbon atoms being preferred. Aralkyl groups can be optionally substituted. Non-limiting examples include, for example, benzyl, diphenylmethyl, triphenylmethyl, phenylethyl, and diphenylethyl.

“Halo” refers to a fluoro, chloro, bromo, or iodo moiety attached to a compound of the invention.

“Heteroaryl” refers to an optionally substituted, mono-, di-, tri- or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl. Heteroaryl may be optionally attached via a carbon or a heteroatom to the rest of the molecule.

“Heteroaralkyl” refers to an optionally substituted, heteroaryl substituted alkyl radicals having from about 2 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

“Perhaloalkyl” refers to an alkyl group, wherein two or more hydrogens are replaced by halo (F, Cl, Br, I) atoms, and alkyl is as previously defined.

Typically, substituted chemical moieties include one or more substituents that replace hydrogen. Exemplary substituents include, for example, halo (e.g., F, Cl, Br, I), alkyl, cycloalkyl, alkylcycloalkyl, alkenyl, alkynyl, aralkyl, aryl, heteroaryl, heteroaralkyl, spiroalkyl, heterocycloalkyl, hydroxyl (—OH), alkoxyl, aryloxyl, aralkoxyl, nitro (—NO2), cyano (—CN), amino (—NH₂), —N-substituted amino (—NHR″), -N,N-disubstituted amino (—N(R″)R″), carboxyl (—COOH), —C(═O)R″, —OR″, —C(═O)OR″, —C(═O)NHSO₂R″, —NHC(═O)R″, aminocarbonyl (—C(═O)NH₂), —N-substituted aminocarbonyl (—C(═O)NHR″), -N,N-disubstituted aminocarbonyl (—C(═O)N(R″)R″), thiol, thiolato (SR″), sulfonic acid and its esters (SO₃R″), phosphonic acid and its mono-ester (P(═O)OR″OH) and di-esters (P(═O)OR″OR″), S(═O)₂R″, S(═O)₂NH₂, S(═O)₂NHR″, S(═O)₂NR″R″, SO₂NHC(═O)R″, NHS(═O)₂R″, NR″S(═O)₂R″, CF₃, CF₂CF₃, NHC(═O)NHR″, NHC(═O)NR″R″, NR″C(═O)NHR″, NR″C(═O)NR″R″, NR″C(═O)R″ and the like. Aryl substituents may also include (CH₂)ᵤSO₂NR″(CH₂)ᵥ and (CH₂)ᵤCO₂NR″(CH₂)ᵥ, where u and v are, independently, 0 to 3, where the methylene units are attached in a 1,2 arrangement yielding substituted aryls of the type:

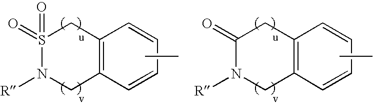

In relation to the aforementioned substituents, each moiety R″ can be, independently, any of H, alkyl, cycloalkyl, alkenyl, aryl, aralkyl, heteroaryl, or heterocycloalkyl, or when (R″(R″)) is attached to a nitrogen atom, R″ and R″ can be taken together to form a 4- to 8-membered nitrogen heterocycle, wherein said heterocycloalkyl ring is optionally interrupted by one or more additional —O—, —S—, —SO, —SO₂—, —NH—, —N(alkyl)-, or —N(aryl)-groups, for example.

“Ligand” or “modulator” refers to a compound that binds to a receptor to form a complex, and specifically includes, agonists, partial agonists, antagonists and inverse agonists.

“Agonist” refers to a compound that binds to a receptor to form a complex that preferably elicits a full pharmacological response, peculiar to the nature of the receptor involved, and preferably alters the equilibrium between inactive and active receptor.

“Partial agonist” refers to a compound that binds to a receptor to form a complex that preferably elicits only a proportion of the full pharmacological response, peculiar to the nature of the receptor involved, even if a high proportion of the receptors are occupied by the compound.

“Antagonist” refers to a compound that binds to a receptor to form a complex that preferably does not elicit any response, in the same manner as an unoccupied receptor, and does not alter the equilibrium between inactive and active receptor.

“Inverse agonist” refers to a compound that binds to a receptor to form a complex that may preferentially stabilize the inactive conformation of the receptor.

“Prodrug” refers to compounds that may serve to maximize the amount of active species that reaches the desired site of reaction that are themselves typically inactive or minimally active for the activity desired, but through biotransformation are converted into biologically active metabolites.

“Stereoisomers” refers to compounds that have identical chemical constitution, but differ as regards the arrangement of the atoms or groups in space.

“N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein throughout can be used or prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both basic nitrogen atom and acidic groups often exist in equilibrium with their zwitterionic forms. Thus, any of the compounds described herein throughout that contain, for example, both basic nitrogen and acidic groups, also include reference to their corresponding zwitterions.

“Effective amount” refers to an amount of a compound as described herein that may be therapeutically effective to inhibit, prevent or treat the symptoms of particular disease, disorder, condition, or side effect. Such diseases, disorders, conditions, and side effects include, but are not limited to, those pathological conditions associated with the binding of δ opioid receptor (for example, in connection with the treatment and/or prevention of pain), wherein the treatment or prevention comprises, for example, agonizing the activity thereof by contacting cells, tissues or receptors with compounds of the present invention. Thus, for example, the term “effective amount,” when used in connection with opioids, or opioid replacements, for example, for the treatment of pain, refers to the treatment and/or prevention of the painful condition. The term “effective amount,” when used in connection with compounds active against gastrointestinal dysfunction, refers to the treatment and/or prevention of symptoms, diseases, disorders, and conditions typically associated with gastrointestinal dysfunction. The term “effective amount,” when used in connection with anti-ileus compounds, refers to the treatment and/or prevention of symptoms, diseases, disorders, and conditions typically associated with ileus. The term “effective amount,” when used in connection with compounds useful in the treatment and/or prevention of urogenital tract disorders, refers to the treatment and/or prevention of symptoms, diseases, disorders, and conditions typically associated with urogenital tract disorders and other related conditions. The term “effective amount,” when used in connection with compounds useful in the treatment and/or prevention of immunomodulatory disorders, refers to the treatment and/or prevention of symptoms, diseases, disorders, and conditions typically associated with immunomodulatory disorders and other related conditions. The term “effective amount,” when used in connection with compounds useful in the treatment and/or prevention of inflammatory disorders, refers to the treatment and/or prevention of symptoms, diseases, disorders, and conditions typically associated with inflammatory disorders and other related conditions. The term “effective amount,” when used in connection with compounds useful in the treatment and/or prevention of respiratory function disorders, refers to the treatment and/or prevention of symptoms, diseases, disorders, and conditions typically associated with respiratory function disorders and other related conditions. The term “effective amount,” when used in connection with compounds useful in the treatment and/or prevention of anxiety, mood disorders, stress-related disorders, and attention deficit hyperactivity disorder, refers to the treatment and/or prevention of symptoms, diseases, disorders, and conditions typically associated with anxiety, mood disorders, stress-related disorders, attention deficit hyperactivity disorder and other related conditions. The term “effective amount,” when used in connection with compounds useful in the treatment and/or prevention of sympathetic nervous system disorders, refers to the treatment and/or prevention of symptoms, diseases, disorders, and conditions typically associated with sympathetic nervous system disorders and other related conditions. The term “effective amount,” when used in connection with compounds useful in the treatment and/or prevention of tussis, refers to the treatment and/or prevention of symptoms, diseases, disorders, and conditions typically associated with tussis and other related conditions. The term “effective amount,” when used in connection with compounds useful in the treatment and/or prevention of motor disorders, refers to the treatment and/or prevention of symptoms, diseases, disorders, and conditions typically associated with motor disorders and other related conditions. The term “effective amount,” when used in connection with compounds useful in the treatment of traumatic injuries of the central nervous system, refers to the treatment and/or prevention of symptoms, diseases, disorders, and conditions typically associated with the central nervous system and other related conditions. The term “effective amount,” when used in connection with compounds useful in the treatment and/or prevention of stroke, cardiac arrhythmia or glaucoma, refers to the treatment and/or prevention of symptoms, diseases, disorders, and conditions typically associated with stroke, cardiac arrhythmia, glaucoma and other related conditions. The term “effective amount,” when used in connection with compounds useful in the treatment and/or prevention of sexual dysfunction, refers to the treatment and/or prevention of symptoms, diseases, disorders, and conditions typically associated with sexual dysfunction and other related conditions. The term “effective amount,” when used in connection with compounds useful in improving organ and cell survival, refers to refers to the maintenance and/or improvement of a minimally-acceptable level of organ or cell survival. The term “effective amount,” when used in connection with compounds useful in the treatment and/or prevention of myocardial infarction, refers to the minimum level of compound necessary to provide cardioprotection after myocardial infarction. The term “effective amount,” when used in connection with compounds useful in the treatment and/or prevention of shock, brain edema, cerebral ischemia, cerebral deficits subsequent to cardiac bypass surgery and grafting, systemic lupus erythematosus, Hodgkin's disease, Sjogren's disease, epilepsy, and rejection in organ transplants and skin grafts, refers to the treatment and/or prevention of symptoms, diseases, disorders, and conditions typically associated with shock, brain edema, cerebral ischemia, cerebral deficits subsequent to cardiac bypass surgery and grafting, systemic lupus erythematosus, Hodgkin's disease, Sjogren's disease, epilepsy, and rejection in organ transplants and skin grafts and other related conditions. The term “effective amount,” when used in connection with compounds useful in the treatment of substance addiction, refers to the treatment of symptoms, diseases, disorders, and conditions typically associated with substance addiction and other related conditions. The term “effective amount,” when used in connection with compounds useful in reducing the need for anesthesia or producing and/or maintaining an anesthetic state, refers to the production and/or maintenance of a minimally acceptable anesthetic state.

“Pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term specifically encompasses veterinary uses.

“In combination with,” “combination therapy,” and “combination products” refer, in certain embodiments, to the concurrent administration to a patient of opioids, an anesthetic agent (inhaled anesthetic, hypnotic, anxiolytic, neuromuscular blocker and opioid) and/or optional ingredients (antibiotics, antivirals, antifungals, anti-inflammatories, anesthetics and mixtures thereof) and the compounds of formula I. When administered in combination, each component may be administered at the same time or sequentially in any order at different points in time. Thus, each component may be administered separately but sufficiently closely in time so as to provide the desired therapeutic effect.

“Dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

“Pain” refers to the perception or condition of unpleasant sensory or emotional experience, associated with actual or potential tissue damage or described in terms of such damage. “Pain” includes, but is not limited to, two broad categories of pain: acute and chronic pain (Buschmann, H.; Christoph, T; Friderichs, E.; Maul, C.; Sundermann, B; eds.; Analgesics, Wiley-VCH, Verlag GMbH & Co. KgaA, Weinheim; 2002; Jain, K. K. “A Guide to Drug Evaluation for Chronic Pain”; Emerging Drugs, 5(2), 241-257(2000)). Non-limiting examples of pain include nociceptive pain, inflammatory pain, visceral pain, somatic pain, neuropathic pain, AIDS pain, cancer pain, phantom pain, and psychogenic pain, and pain resulting from hyperalgesia, pain caused by rheumatoid arthritis, migraine, allodynia and the like.

“Gastrointestinal dysfunction” refers collectively to maladies of the stomach, small and large intestine. Non-limiting examples of gastrointestinal dysfunction include, for example, diarrhea, nausea, emesis, post-operative emesis, opioid-induced emesis, irritable bowel syndrome, opioid-bowel dysfunction, post-operative ileus, opioid-induced ileus, colitis, decreased gastric motility, decreased gastric emptying, inhibition of small intestinal propulsion, inhibition of large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, distension, abdominal or epigastric pain and discomfort, non-ulcerogenic dyspepsia, gastritis, constipation, or delayed absorption of orally administered medications or nutritive substances.

“Ileus” refers to the obstruction of the bowel or gut, especially the colon. See, e.g., Dorland's Illustrated Medical Dictionary, p. 816, 27th ed. (W. B. Saunders Company, Philadelphia 1988). Ileus should be distinguished from constipation, which refers to infrequent or difficulty in evacuating the feces. See, e.g., Dorland's Illustrated Medical Dictionary, p. 375, 27th ed. (W. B. Saunders Company, Philadelphia 1988). Ileus may be diagnosed by the disruption of normal coordinated movements of the gut, resulting in failure of the propulsion of intestinal contents. See, e.g., Resnick, J. Am. J. of Gastroenterology 1997, 92, 751 and Resnick, J. Am. J. of Gastroenterology, 1997, 92, 934. In some instances, particularly following surgery, including surgery of the abdomen, the bowel dysfunction may become quite severe, lasting for more than a week and affecting more than one portion of the GI tract. This condition is often referred to as post-surgical (or post-operative) paralytic ileus and most frequently occurs after laparotomy (see Livingston, E. H. and Passaro, E. D. Jr. Digestive Diseases and Sciences 1990, 35, 121). Similarly, post-partum ileus is a common problem for women in the period following childbirth, and is thought to be caused by similar fluctuations in natural opioid levels as a result of birthing stress.

“Urogenital tract disorders” refers collectively to maladies of the urinary and genital apparati. Non-limiting examples of urogenital tract disorders include incontinence.

“Immunomodulatory disorders” refers collectively to maladies characterized by a compromised or over-stimulated immune system. Non-limiting examples of immunomodulatory disorders include an autoimmune disease (such as arthritis, an autoimmune disorder associated with a skin graft, an autoimmune disorder associated with organ transplant, and an autoimmune disorder associated with surgery), a collagen disease, an allergy, a side effect associated with the administration of an anti-tumor agent, and a side effect associated with the administration of an antiviral agent.

“Inflammatory disorders” refers collectively to maladies characterized by cellular events in injured tissues. Non-limiting examples of inflammatory diseases include arthritis, psoriasis, asthma, and inflammatory bowel disease.

“Respiratory function disorders” refers to conditions in which breathing and/or airflow into the lung is compromised. Non-limiting examples of respiratory function disorders include asthma, apnea, tussis, and lung edema.

“Lung edema” refers to the presence of abnormally large amounts of fluid in the intercellular tissue spaces of the lungs.

“Anxiety” refers to the unpleasant emotional state consisting of psychophysiological responses to anticipation of unreal or imagined danger, ostensibly resulting from unrecognized intrapsychic conflict.

“Mood disorders” refers to disorders that have a disturbance in mood as their predominant feature, including depression, bipolar manic-depression, and seasonal affective disorder.

“Depression” refers to a mental state of depressed mood characterized by feelings of sadness, despair and discouragement, including the blues, dysthymia, and major depression.

“Stress-related disorders” refer collectively to maladies characterized by a state of hyper- or hypoarousal with hyper- and hypovigilance. Non-limiting examples of stress-related disorders include post-traumatic stress disorder, panic disorder, generalized anxiety disorder, social phobia, and obsessive-compulsive disorder.

“Attention deficit hyperactivity disorder” refers to a condition characterized by an inability to control behavior due to difficulty in processing neural stimuli.

“Sympathetic nervous system disorders” refer collectively to maladies characterized by disturbances of the autonomic nervous system. Non-limiting examples of sympathetic nervous system disorders include hypertension, and the like.

“Tussis” refers to a coughing condition, and “antitussive” agents refer to those materials that modulate the coughing response.

“Motor disorders” refers to involuntary manifestations of hyper or hypo muscle activity and coordination. Non-limiting examples of motor disorders include tremors, Parkinson's disease, and Tourette syndrome.

“Traumatic injury of the central nervous system” refers to a physical wound or injury to the spinal cord or brain.

“Stroke” refers to a condition due to the lack of oxygen to the brain.

“Cardiac arrhythmia” refers to a condition characterized by a disturbance in the electrical activity of the heart that manifests as an abnormality in heart rate or heart rhythm. Patients with a cardiac arrhythmia may experience a wide variety of symptoms ranging from palpitations to fainting.

“Glaucoma” refers collectively to eye diseases characterized by an increase in intraocular pressure that causes pathological changes in the optic disk and typical defects in the field of vision.

“Sexual dysfunction” refers collectively to disturbances, impairments or abnormalities of the functioning of the male or female sexual organs, including, but not limited to premature ejaculation.

“Cardioprotection” refers to conditions or agents that protect or restore the heart from dysfunction.

“Myocardial infarction” refers to irreversible injury to heart muscle caused by a local lack of oxygen.

“Addiction” refers to a pattern of compulsive substance abuse (alcohol, nicotine, or drug) characterized by a continued craving for the substance and, in some cases, the need to use the substance for effects other than its prescribed or legal use.

“Anesthetic state” refers to the state of the loss of feeling or sensation, including not only the loss of tactile sensibility or of any of the other senses, but also to the loss of sensation of pain, as it is induced to permit performance of surgery or other painful procedures, and specifically including amnesia, analgesia, muscle relaxation and sedation.

“Patient” refers to animals, including mammals, preferably humans.

“Side effect” refers to a consequence other than the one(s) for which an agent or measure is used, as the adverse effects produced by a drug, especially on a tissue or organ system other then the one sought to be benefited by its administration. In the case, for example, of opioids, the term “side effect” may refer to such conditions as, for example, constipation, nausea and/or vomiting.

When any variable occurs more than one time in any constituent or in any formula, its definition in each occurrence is independent of its definition at every other occurrence. Combinations of substituents and/or variables are permissible only if such combinations result in stable compounds.

It is believed the chemical formulas and names used herein correctly and accurately reflect the underlying chemical compounds. However, the nature and value of the present invention does not depend upon the theoretical correctness of these formulae, in whole or in part. Thus it is understood that the formulas used herein, as well as the chemical names attributed to the correspondingly indicated compounds, are not intended to limit the invention in any way, including restricting it to any specific tautomeric form or to any specific optical or geometric isomer, except where such stereochemistry is clearly defined.

In certain preferred embodiments, the compounds, pharmaceutical compositions and methods of the present invention may involve a peripheral δ opioid modulator compound. The term “peripheral” designates that the compound acts primarily on physiological systems and components external to the central nervous system. In preferred form, the peripheral δ opioid modulator compounds employed in the methods of the present invention exhibit high levels of activity with respect to peripheral tissue, such as, gastrointestinal tissue, while exhibiting reduced, and preferably substantially no, CNS activity. The phrase “substantially no CNS activity,” as used herein, means that less than about 50% of the pharmacological activity of the compounds employed in the present methods is exhibited in the CNS, preferably less than about 25%, more preferably less than about 10%, even more preferably less than about 5% and most preferably 0% of the pharmacological activity of the compounds employed in the present methods is exhibited in the CNS.

Furthermore, it is preferred in certain embodiments of the invention that the δ opioid modulator compound does not substantially cross the blood-brain barrier. The phrase “does not substantially cross,” as used herein, means that less than about 20% by weight of the compound employed in the present methods crosses the blood-brain barrier, preferably less than about 15% by weight, more preferably less than about 10% by weight, even more preferably less than about 5% by weight and most preferably 0% by weight of the compound crosses the blood-brain barrier. Selected compounds can be evaluated for CNS penetration by determining plasma and brain levels following i.v. administration.

Accordingly, in one embodiment, the invention is directed to compounds of formula I: 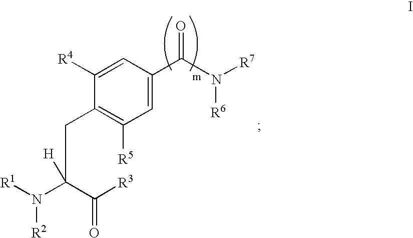
or a stereoisomer, prodrug, pharmaceutically acceptable salt, hydrate, solvate, acid salt hydrate, N-oxide or isomorphic crystalline form thereof.

In formula I, R¹ and R² are each, independently, H, alkyl, alkenyl, or —C(═NH)NH₂, provided that at least one of R¹ and R² is other than —C(═NH)NH₂, or R¹ and R² taken together with the nitrogen atom to which they are attached form a 4- to 8-membered heterocycloalkyl ring wherein the heterocycloalkyl ring is optionally interrupted with one additional O, N, or S heteroatom. Preferably, R¹ and R² are each, independently, H or alkyl, and more preferably, R¹ and R² are each H.

In formula I, R³ is -[J]ₒ-X,

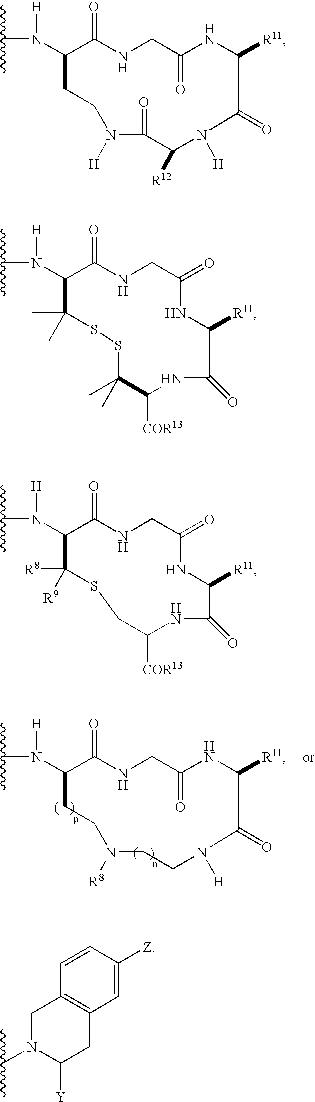

In certain preferred embodiments, R³ is:
-[arg-Phe-Lys]-X,
-[Pro-Trp-Phe]-X,
-[Gly-Gly-Phe-Leu]-X,
-[ala-Gly-Phe-Leu]-X,
-[ala-Gly-N(CH₃)Phe]-X,
-[ser-Gly-Phe-Leu-Thr]-X,
-[ala-Phe-Gly-Tyr-Pro-Ser]-X, or
-[Gly-Gly-Phe-Leu-Arg-Arg-Ile-Arg-Pro-Lys]-X.

In other preferred embodiments R³ is:

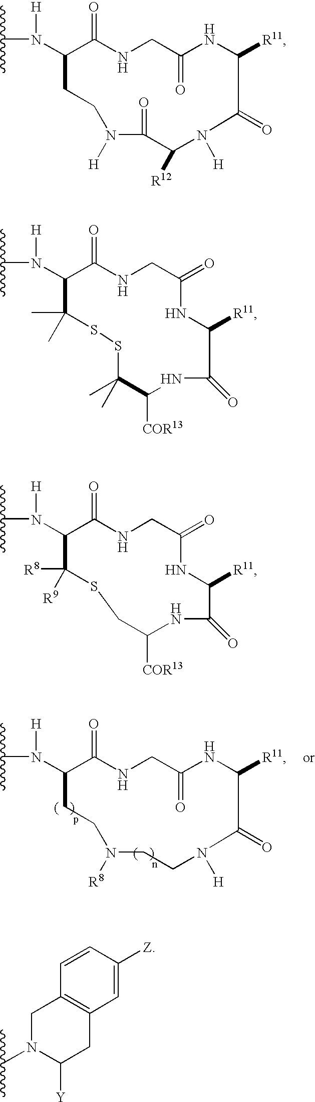

In certain more preferred embodiments, R³ is:

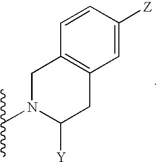

In certain more preferred embodiments, R³ is:

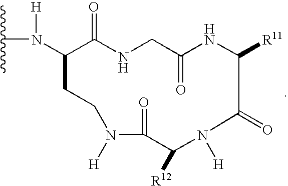

In certain more preferred embodiments, R³ is:

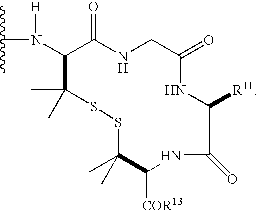

In certain more preferred embodiments, R³ is:

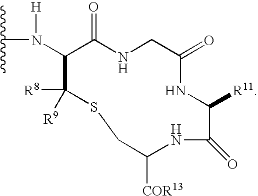

In certain more preferred embodiments, R³ is:

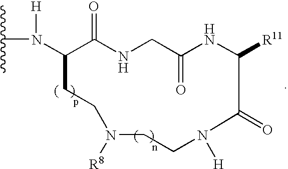

In formula I, J is [NR^{8a}(C(R^{9a})(R¹⁰))ₙ—C(═O)]. In certain preferred embodiments, each J is, independently, the amino acid glycine, phenylalanine, leucine, alanine, arginine, lysine, isoleucine, proline, tryptophan, serine, methionine, threonine, tyrosine, histidine, or (N-methyl)phenylalanine or combinations thereof. In certain more preferred embodiments, each J is, independently, the amino acid phenylalanine, arginine, lysine, proline, or tryptophan or combinations thereof. In certain other more preferred embodiments, each J is, independently, the amino acid glycine, phenylalanine, leucine, or alanine or combinations thereof. In other more preferred embodiments, each J is, independently, the amino acid serine, glycine, phenylalanine, leucine, or threonine or combinations thereof.

In formula I, X is OR⁸, NR^{8b}R^{9b}, or —N(R^{8c})CH(R^{9c})(R¹⁰). Preferably, X is —OH, —NH₂, or —NHCH₂CH₂OH. More preferably, X is —OH or —NH₂. Even more preferably, X is —NH₂.

In formula I, R⁴ and R⁵ are each, independently, H or alkyl. Preferably, R⁴ and R⁵ are each, independently, H or methyl.

In formula I, R⁶ and R⁷ are each, independently, H or alkyl, or R⁶ and R⁷ taken together with the nitrogen atom to which they are attached form a 4- to 8-membered heterocycloalkyl ring wherein the heterocycloalkyl ring is optionally interrupted with one additional O, N, or S heteroatom. Preferably, R and R⁷ are each, independently, H or alkyl, preferably H.

In certain preferred embodiments, R¹, R², R⁴, R⁵, R⁶, and R⁷ are each H.

In formula I, R⁸ and R⁹ are each, independently, H or alkyl. In certain more preferred embodiments, R⁸ and R⁹ are each, hydrogen. In certain more preferred embodiments, R⁸ and R⁹ are each, methyl.

In formula I, R^{8a} and R^{9a} are each, independently, H or alkyl, or R^{8a} and R^{9a} taken together with the atoms through which they are connected form a 4- to 14-membered heterocycloalkyl ring wherein the heterocycloalkyl ring is optionally interrupted with one additional O, N, or S heteroatom. In certain preferred embodiments, R^{8a} and R^{9a} are each, independently, H or alkyl. More preferably, R^{8a} and R^{9a} are H. In other preferred embodiments, R^{8a} and R^{9a} taken together with the nitrogen and carbon atoms through which they are connected form a 4- to 14-membered heterocycloalkyl ring wherein the heterocycloalkyl ring is optionally interrupted with one additional O, N, or S heteroatom.

In formula I, R^{8b} and R^{9b} are each, independently, H or alkyl, or R^{8b} and R^{9b} taken together with the nitrogen atom to which they are attached form a 4- to 8-membered heterocycloalkyl ring wherein the heterocycloalkyl ring is optionally interrupted with one additional O, N, or S heteroatom. In certain preferred embodiments, R^{8b} and R^{9b} are each, independently, H or alkyl.

In formula I, R^{8c} and R^{9b} are each, independently, H or alkyl, or R^{8c} and R^{9c} taken together with the nitrogen and carbon atoms through which they are connected form a 4- to 8-membered heterocycloalkyl ring, wherein the heterocycloalkyl ring is optionally interrupted with one additional O, N, or S heteroatom, provided that the additional heteroatom is separated by at least two carbon atoms from the nitrogen atom to which R^{8c} is attached. R^{8b} and R^{9b} are each, independently, H or alkyl.

In formula I, R¹⁰ is —(C(R¹⁴)(R¹⁵))_{q}—R¹⁶, preferably R¹⁰ is H.

In formula I, R″ and R 2 are each, independently, H or —C(R¹⁴)(R¹⁵)R¹⁰. In preferred embodiments, R¹¹ is aralkyl. In more preferred embodiments, R¹¹ is benzyl. In preferred embodiments, R¹² is alkyl. In more preferred embodiments, R¹² is 2-methylprop-1-yl.

In formula I, R¹³ is —OR¹⁴ or —NR¹⁴R¹⁵, preferably, R¹³—OH or NH₂, more preferably, R¹³ is —OH.

In formula I, R¹⁴ and R¹⁵ are each, independently, H or alkyl, preferably H.

In formula I, R¹⁶ is H, alkyl, cycloalkyl, heterocycloalkyl, aryl, heteroaryl, —CR¹⁴R¹⁵OH, —CR¹⁴R¹⁵S(O)ᵣR⁹, —CR¹⁴R¹⁵COOR⁴, —CR¹⁴R¹⁵CONHR⁴, CR¹⁴R¹⁵NHR⁴, or —CR¹⁴R¹⁵NH(C═NH)NH₂. Preferably, R¹⁶ is H or alkyl. More preferably, R¹⁶ is H.

In formula I, Y is heteroaryl, preferably imidazol-2-yl or oxazol-2-yl.

In formula I, Z is H or —OR⁸, preferably H or —OH.

In formula I, m is the integer 0 or 1, preferably, m is 1.

In formula I, n is the integer from 1 to 4, preferably n is 1.

In formula I, o is an integer from 0 to 16, preferably 1 to 10, more preferably 1 to 6, and even more preferably 1 to 4.

In formula I, p is the integer from 0 to 3, preferably, p is 1.

In formula I, q is an integer from 0 to 6, preferably, q is 0 to 3, more preferably, q is 0-2.

In formula I, r is the integer 0 to 2, preferably, r is 0.

In certain embodiments, the compounds of the invention are of formula Ia:

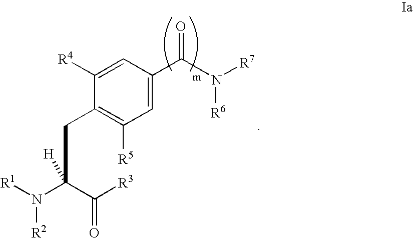

In certain preferred embodiments of the invention, R³ is:
[NR^{8d}(C(R^{9d})(R¹⁰))ₙ—C(═O)ₛ-J-[NR^{8e}(C(R^{9e})(R¹⁰))ₙ—C(═O)]ₜ—X;
wherein:
R^{8d} and R^{9d} are each, independently, H or alkyl, or R^{8d} taken together with the R^{9d} on the carbon atom alpha to the nitrogen bearing the R^{8d} and the nitrogen and carbon atoms through which they are connected form a 4- to 14-membered heterocycloalkyl ring wherein the heterocycloalkyl ring is optionally interrupted with one additional O, N, or S heteroatom;
R^{8e} and R^{9e} are each, independently, H or alkyl, or R^{8e} taken together with the R^{9e} on the carbon atom alpha to the nitrogen bearing the R^{8e} and the nitrogen and carbon atoms through which they are connected form a 4- to 8-membered heterocycloalkyl ring wherein the heterocycloalkyl ring is optionally interrupted with one additional O, N, or S heteroatom; and
the sum of s+t is an integer of 0 to 9.

In preferred embodiments, the sum of s+t is 0 to 2, more preferably, the sum of s+t is 0 or 1, and even more preferably, the sum of s+t is 0.

In certain preferred embodiments, J is [NR^{8a}(CH(R^{9a})ₙ—C(═O)]. Preferably, n is 1. Preferably, R^{8a} is H. Preferably, R^{9a} is H, benzyl, —(CH₂)₄—NH₂, CH₂-indol-3-yl, —(CH₂)₃—NH—C(═NH)NH₂, —CH₂CH(CH₃)₂, —CH₃, —CH₂OH, —CH(OH)CH₃, (para-hydroxyphenyl)methyl-, or —CH(CH₃)CH₂CH₃, or R^{8a} and R^{9a} taken together with the atoms through which they are connected form a 4- to 14-membered heterocycloalkyl ring, wherein the heterocycloalkyl ring is optionally interrupted with one additional O, N, or S heteroatom. More preferably, R^{9a} is benzyl, —(CH₂)₄—NH₂, CH₂-indol-3-yl, or —(CH₂)₃—NH—C(═NH)NH₂, or R^{8a} and R^{9a} taken together with the atoms through which they are connected form a 4- to 8-membered heterocycloalkyl ring wherein the heterocycloalkyl ring is optionally interrupted with one additional O, N, or S heteroatom.

In certain preferred embodiments, the compounds of the invention are of formula II:

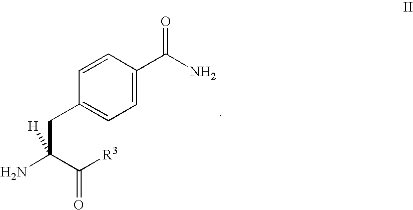

Particularly preferred carboxamide and amino derivatives of the invention include:
H-(S)-2-Amino-3-(4-carboxamidophenyl)propionic acid-Gly-Gly-Phe-Leu-NH;
H-(S)-2-Amino-3-(4-carboxamidophenyl)propionic acid-Gly-Gly-Phe-Leu;
H-(S)-2-Amino-3-(4-carboxamidophenyl)propionic acid-ala-Gly-Phe-leu;
H-(S)-2-Amino-3-(4-carboxamidophenyl)propionic acid-ala-Gly-Phe-leu-NH₂;
H-(S)-2-Amino-3-(4-carboxamidophenyl)propionic acid-Gly-Gly-Phe-leu-NH₂;
H-(S)-2-Amino-3-(4-carboxamidophenyl)propionic acid-Arg-Phe-Lys-NH₂;
H-(S)-2-Amino-3-(4-carboxamidophenyl)propionic acid-arg-Phe-Lys-NH₂;
H-(S)-2-Amino-3-(4-carboxamidophenyl)propionic acid-Pro-Trp-Phe-NH₂;
H-(S)-2-Amino-3-(4-carboxamidophenyl)propionic acid-ala-Phe-Gly-Tyr-Pro-Ser;
H-(S)-2-Amino-3-(4-carboxamidophenyl)propionic acid-met-Phe-His-Leu-Met-Asp;
H-(S)-2-Amino-3-(4-carboxamidophenyl)propionic acid-ser-Gly-Phe-Leu-Thr;
H-(S)-2-Amino-3-(4-carboxamidophenyl)propionic acid-Gly-Gly-Phe-Leu-Arg-Arg-Ile-Arg-Pro-Lys;
H-(S)-2-Amino-3-(4-carboxamidophenyl)propionic acid-ala-Gly-N(Me)Phe-NHCH₂CH₂OH;
(S)-2-Amino-3-(4-carboxamidophenyl)propionic acid-c[D-A₂bu-Gly-Phe-Leu];
(S)-2-Amino-3-(4-carboxamidophenyl)propionic acid-c[D-Val_{L}-Gly-Phe-D-Ala_{L}]-OH; 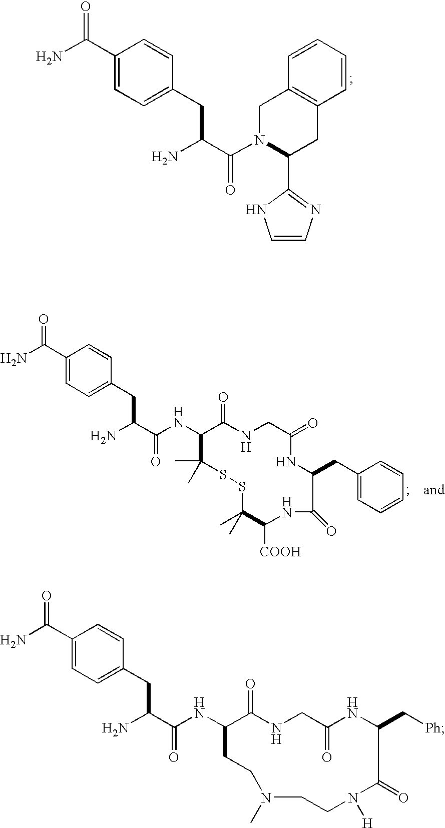
   or a stereoisomer, prodrug, pharmaceutically acceptable salt, hydrate, solvate, acid salt hydrate, N-oxide or isomorphic crystalline form thereof.

Compounds of the invention are useful as analgesic agent for use during general anesthesia and monitored anesthesia care. Combinations of agents with different properties are often used to achieve a balance of effects needed to maintain the anesthetic state (e.g., amnesia, analgesia, muscle relaxation and sedation). Included in this combination are inhaled anesthetics, hypnotics, anxiolytics, neuromuscular blockers and opioids.

In any of the above teachings, a compound of the invention may be either a compound of one of the formulae herein described, or a stereoisomer, prodrug, pharmaceutically acceptable salt, hydrate, solvate, acid salt hydrate, N-oxide or isomorphic crystalline form thereof.

The compounds employed in the methods of the present invention may exist in prodrug form. As used herein, “prodrug” is intended to include any covalently bonded carriers which release the active parent drug, for example, as according to formula I or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example formula I, may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

Compounds employed in the present methods may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

The compounds employed in the methods of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include the benzyloxycarbonyl group and the tert-butyloxycarbonyl group. Other preferred protecting groups that may be employed in accordance with the present invention may be described in Greene, T. W. and Wuts, P. G. M., Protective Groups in Organic Synthesis 2d. Ed., Wiley & Sons, 1991.

The δ agonist compounds employed in the methods of the present invention may be administered by any means that results in the contact of the active agent with the agent's site of action in the body of a patient. The compounds may be administered by any conventional means available for use in conjunction with pharmaceuticals, either as individual therapeutic agents or in a combination of therapeutic agents. For example, they may be administered as the sole active agent in a pharmaceutical composition, or they can be used in combination with other therapeutically active ingredients including, for example, opioid analgesic agents. In such combinations, selected compounds of the invention may provide equivalent or even enhanced therapeutic activity such as, for example, pain ameliorization, while providing reduced adverse side effects associated with opioids, such as addiction or pruritus, by lowering the amount of opioid required to achieve a therapeutic effect.

The compounds are preferably combined with a pharmaceutical carrier selected on the basis of the chosen route of administration and standard pharmaceutical practice as described, for example, in Remington 's Pharmaceutical Sciences (Mack Publishing Co., Easton, Pa., 1980), the disclosures of which are hereby incorporated herein by reference, in their entirety.

In addition to the pharmaceutical carrier, the compounds of formula I may be co-administered with at least one opioid, preferably a μ opioid receptor modulator compound. In certain embodiments, the combination of the compounds of formula I with at least one opioid, preferably a μ opioid receptor modulator compound, provides a synergistic analgesic effect. The utility of the instant combination product may be determined by those skilled in the art using established animal models. Suitable opioids include, without limitation, alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine (pethidine), meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, diastereoisomers thereof, pharmaceutically acceptable salts thereof, complexes thereof; and mixtures thereof.

The opioid component of the present compositions may further include one or more other active ingredients that may be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosure of which is hereby incorporated herein by reference, in its entirety.

In addition, the opioid component may further include one or more compounds that may be designed to enhance the analgesic potency of the opioid and/or to reduce analgesic tolerance development. Such compounds include, for example, dextromethorphan or other NMDA antagonists (Mao, M. J. et al., Pain 1996, 67, 361), L-364,718 and other CCK antagonists (Dourish, C. T. et al., Eur J Pharmacol 1988, 147, 469), NOS inhibitors (Bhargava, H. N. et al., Neuropeptides 1996, 30, 219), PKC inhibitors (Bilsky, E. J. et al., J Pharmacol Exp Ther 1996, 277, 484), and dynorphin antagonists or antisera (Nichols, M. L. et al., Pain 1997, 69, 317). The disclosures of each of the foregoing documents are hereby incorporated herein by reference, in their entireties.

Other opioids, optional conventional opioid components, and optional compounds for enhancing the analgesic potency of the opioid and/or for reducing analgesic tolerance development, that may be employed in the methods and compositions of the present invention, in addition to those exemplified above, would be readily apparent to one of ordinary skill in the art, once armed with the teachings of the present disclosure.

Compounds of the present invention can be administered to a mammalian host in a variety of forms adapted to the chosen route of administration, e.g., orally or parenterally. Parenteral administration in this respect includes administration by the following routes: intravenous, intramuscular, subcutaneous, rectal, intraocular, intrasynovial, transepithelial including transdermal, ophthalmic, sublingual and buccal; topically including ophthalmic, dermal, ocular, rectal, and nasal inhalation via insufflation aerosol.

The active compound may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparations should preferably contain at least 0.1% of active compound. The percentage of the compositions and preparations may, of course, be varied and may conveniently be, for example, from about 2 to about 6% of the weight of the unit. The amount of active compound in such therapeutically useful compositions is preferably such that a suitable dosage will be obtained. Preferred compositions or preparations according to the present invention may be prepared so that an oral dosage unit form contains from about 0.1 to about 1000 mg of active compound.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compound as a free base or a pharmacologically acceptable salt can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compound in the required amount, in the appropriate solvent, with various of the other ingredients enumerated above, as required, followed by filtered sterilization. Generally, dispersions may be prepared by incorporating the sterilized active ingredient into a sterile vehicle that contains the basic dispersion medium and the required other ingredients from those enumerated above. In the case of sterile powders for the preparation of sterile injectable solutions, the preferred methods of preparation may include vacuum drying and the freeze-drying technique that yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compound, chosen route of administration and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. The therapeutic human dosage, based on physiological studies using rats, may generally range from about 0.01 mg to about 100 mg/kg of body weight per day, and all combinations and subcombinations of ranges therein. Alternatively, the therapeutic human dosage may be from about 0.4 mg to about 10 g or higher, and may be administered in several different dosage units from once to several times a day. Generally speaking, oral administration may require higher dosages.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The dose may also be provided by controlled release of the compound, by techniques well known to those in the art.

The compounds of the invention may also be formulated with other optional active ingredients, in addition to the optional opioids, and in addition to the optional pharmaceutical-acceptable carriers. Other active ingredients include, but are not limited to, antibiotics, antivirals, and antifungals, anti-inflammatories, including steroidal and non-steroidal anti-inflammatories, anesthetics, and mixtures thereof. Such additional ingredients include any of the following:

a. Antibacterial Agents
Aminoglycosides, such as Amikacin, Apramycin, Arbekacin, Bambermycins, Butirosin, Dibekacin, Dihydrostreptomycin, Fortimicin(s), Fradiomycin, Gentamicin, Ispamicin, Kanamycin, Micronomicin, Neomycin, Neomycin Undecylenate, Netilmicin, Paromomycin, Ribostamycin, Sisomicin, Spectinomycin, Streptomycin, Streptonicozid and Tobramycin;
Amphenicols, such as Azidamfenicol, Chloramphenicol, Chloramphenicol Palmirate, Chloramphenicol Pantothenate, Florfenicol, Thiamphenicol;
Ansamycins, such as Rifamide, Rifampin, Rifamycin and Rifaximin;
γ-Lactams;
Carbapenems, such as Imipenem;
Cephalosporins, such as 1-Carba (dethia) Cephalosporin, Cefactor, Cefadroxil, Cefamandole, Cefatrizine, Cefazedone, Cefazolin, Cefixime, Cefinenoxime, Cefodizime, Cefonicid, Cefoperazone, Ceforanide, Cefotaxime, Cefotiam, Cefpimizole, Cefpirimide, Cephodoxime Proxetil, Cefroxadine, Cefsulodin, Ceftazidime, Cefteram, Ceftezole, Ceftibuten, Ceftizoxime, Ceftriaxone, Cefuroxime, Cefuzonam, Cephacetrile Sodium, Cephalexin, Cephaloglycin, Cephaloridine, Cephalosporin, Cephalothin, Cephapirin Sodium, Cephradine and Pivcefalexin;
Cephamycins such as Cefbuperazone, Cefmetazole, Cefminox, Cefetan and Cefoxitin;
Monobactams such as Aztreonam, Carumonam and Tigemonan;
Oxacephems such as Flomoxef and Moxolactam;
Penicillins such as Amidinocillin, Amdinocillin, Pivoxil, Amoxicillin, Ampicillan, Apalcillin, Aspoxicillin, Azidocillan, Azlocillan, Bacampicillin, Benzylpenicillinic Acid, Benzylpenicillin, Carbenicillin, Carfecillin, Carindacillin, Clometocillin, Cloxacillin, Cyclacillin, Dicloxacillin, Diphenicillin, Epicillin, Fenbenicillin, Floxicillin, Hetacillin, Lenampicillin, Metampicillin, Methicillin, Mezlocillin, Nafcillin, Oxacillin, Penamecillin, Penethamate Hydriodide, Penicillin G Benethamine, Penicillin G Benzathine, Penicillin G Benzhydrylamine, Penicillin G Calcium, Penicillin G Hydragamine, Penicillin G Potassium, Penicillin G. Procaine, Penicillin N, Penicillin 0, Penicillin V, Penicillin V Benzathine, Penicillin V Hydrabamine, Penimepicycline, Phenethicillin, Piperacillin, Pivapicillin, Propicillin, Quinacillin, Sulbenicillin, Talampicillin, Temocillin and Ticarcillin;
Lincosumides such as Clindamycin and Lincomycin;
Macrolides such as Azithromycin, Carbomycin, Clarithromycin, Erythromycin(s) and Derivatives, Josamycin, Leucomycins, Midecamycins, Miokamycin, Oleandomycin, Primycin, Rokitamycin, Rosaramicin, Roxithromycin, Spiramycin and Troleandomycin;
Polypeptides such as Amphomycin, Bacitracin, Capreomycin, Colistin, Enduracidin, Enviomycin, Fusafungine, Gramicidin(s), Gramicidin S, Mikamycin, Polymyxin, Polymyxin β-Methanesulfonic Acid, Pristinamycin, Ristocetin, Teicoplanin, Thiostrepton, Tuberactinomycin, Tyrocidine, Tyrothricin, Vancomycin, Viomycin(s), Virginiamycin and Zinc Bacitracin;
Tetracyclines such as Spicycline, Chlortetracycline, Clomocycline, Demeclocycline, Doxycycline, Guamecycline, Lymecycline, Meclocycline, Methacycline, Minocycline, Oxytetracycline, Penimepicycline, Pipacycline, Rolitetracycline, Sancycline, Senociclin and Tetracycline; and
others such as Cycloserine, Mupirocin, Tuberin.

b. Synthetic Antibacterials
2,4-Diaminopyrimidines such as Brodimoprim, Tetroxoprim, and Trimethoprim;
Nitrofurans such as Furaltadone, Furazolium, Nifuradene, Nifuratel, Nifurfoline, Nifurpirinol, Nifirprazine, Nifurtoinol and Nitrofurantoin;
Quinolones and analogs thereof, such as Amifloxacin, Cinoxacin, Ciprofloxacin, Difloxacin, Enoxacin, Fleroxacin, Flumequine, Lomefloxacin, Miloxacin, Nalidixic Acid, Norfloxacin, Ofloxacin, Oxolinic Acid, Perfloxacin, Pipemidic Acid, Piromidic Acid, Rosoxacin, Temafloxacin and Tosufloxacin;
Sulfonamides such as Acetyl Sulfamethoxypyrazine, Acetyl Sulfisoxazole, Azosulfamide, Benzylsulfamide, Chloramine-β, Chloramine-T, Dichloramine-T, Formosulfathiazole, N.sup.2-Formyl-sulfisomidine, N.sup.4-β-D-Glucosylsulfanilamide, Mafenide, 4′-(Methyl-sulfamoyl)sulfanilanilide, p-Nitrosulfathiazole, Noprylsulfamide, Phthalylsulfacetamide, Phthalylsulfathiazole, Salazosulfadimidine, Succinylsulfathiazole, Sulfabenzamide, Sulfacetamide, Sulfachlorpyridazine, Sulfachrysoidine, Sulfacytine, Sulfadiazine, Sulfadicramide, Sulfadimethoxine, Sulfadoxine, Sulfaethidole, Sulfaguanidine, Sulfaguanol, Sulfalene, Sulfaloxic Acid, Sulfamerazine, Sulfameter, Sulfamethazine, Sulfamethizole, Sulfamethomidine, Sulfamethoxazole, Sulfamethoxypyridazine, Sulfametrole, sulfamidochrysoidine, Sulfamoxole, Sulfanilamide, Sulfanilamidomethanesulfonic Acid Triethanolamine Salt, 4-Sulfanilamidosalicyclic Acid, N⁴-Sulfanilylsulfanilamide, Sulfanilylurea, N-Sulfanilyl-3,4-xylamide, Sulfanitran, Sulfaperine, Sulfaphenazole, Sulfaproxyline, Sulfapyrazine, Sulfapyridine, Sulfasomizole, Sulfasymazine, Sulfathiazole, Sulfathiourea, Sulfatolamide, Sulfisomidine and Sulfisoxazole;
Sulfones, such as Acedapsone, Acediasulfone, Acetosulfone, Dapsone, Diathymosulfone, Glucosulfone, Solasulfone, Succisulfone, Sulfanilic Acid, p-Sulfanilylbenzylamine, p,p′-sulfonyldianiline-N,N′-digalactoside, Sulfoxone and Thiazolsulfone;
Others such as Clofoctol, Hexedine, Magainins, Methenamine, Methenamine Anhydromethylene-citrate, Methenamine Hippurate, Methenamine Mandelate, Methenamine Sulfosalicylate, Nitroxoline, Squalamine and Xibomol.

c. Antifungal (Antibiotics)

Polyenes such as Amphotericin-B, Candicidin, Dermostatin, Filipin, Fungichromin, Hachimycin, Hamycin, Lucensomycin, Mepartricin, Natamycin, Nystatin, Pecilocin, Perimycin; and others, such as Azaserine, Griseofulvin, Oligomycins, PyrroInitrin, Siccanin, Tubercidin and Viridin.

d. Antifungal (Synthetic)
Allylamines such as Naflifine and terbinafine;
Imidazoles such as Bifonazole, Butoconazole, Chlordantoin, Chlormidazole, Cloconazole, Clotrimazole, Econazole, Enilconazole, Finticonazole, Isoconazole, Ketoconazole, Miconazole, Omoconazole, Oxiconazole Nitrate, Sulconazole and Tioconazole;
Triazoles such as Fluconazole, Itraconazole, Terconazole;
Others such as Acrisorcin, Amorolfine, Biphenamine, Bromosalicylchloranilide, Buclosamide, Chlophenesin, Ciclopirox, Cloxyquin, Coparaffinate, Diamthazole, Dihydrochloride, Exalamide, Flucytosine, Halethazole, Hexetidine, Loflucarban, Nifuratel, Potassium Iodide, Propionic Acid, Pyrithione, Salicylanilide, Sulbentine, Tenonitrozole, Tolciclate, Tolindate, Tolnaftate, Tricetin, Ujothion, and Undecylenic Acid.

e. Antiglaucoma Agents

Antiglaucoma agents, such as Dapiprazoke, Dichlorphenamide, Dipivefrin, and Pilocarpine.

f. Anti-Inflammatory Agents
Corticosteroids, aminoarylcarboxylic Acid Derivatives such as Etofenamate, Meclofenamic Acid, Mefanamic Acid, Niflumic Acid;
Arylacetic Acid Derivatives such as Acemetacin, Amfenac Cinmetacin, Clopirac, Diclofenac, Fenclofenac, Fenclorac, Fenclozic Acid, Fentiazac, Glucametacin, Isozepac, Lonazolac, Metiazinic Acid, Oxametacine, Proglumetacin, Sulindac, Tiaramide and Tolmetin;
Arylbutyric Acid Derivatives such as Butibufen and Fenbufen;
Arylcarboxylic Acids such as Clidanac, Ketorolac and Tinoridine;
Arylpropionic Acid Derivatives such as Bucloxic Acid, Carprofen, Fenoprofen, Flunoxaprofen, Ibuprofen, Ibuproxam, Oxaprozin, Piketoprofen, Pirprofen, Pranoprofen, Protizinic Acid and Tiaprofenic Add;
Pyrazoles such as Mepirizole;
Pyrazolones such as Clofezone, Feprazone, Mofebutazone, Oxyphenbutazone, Phenylbutazone, Phenyl Pyrazolidininones, Suxibuzone and Thiazolinobutazone;
Salicylic Acid Derivatives such as Bromosaligenin, Fendosal, Glycol Salicylate, Mesalamine, 1-Naphthyl Salicylate, Olsalazine and Sulfasalazine;
Thiazinecarboxamides such as Droxicam, Isoxicam and Piroxicam;
Others such as e-Acetamidocaproic Acid, S-Adenosylmethionine, 3-Amino-4-hydroxybutyric Acid, Amixetrine, Bendazac, Bucolome, Carbazones, Difenpiramide, Ditazol, Guaiazulene, Heterocyclic Aminoalkyl Esters of Mycophenolic Acid and Derivatives, Nabumetone, Nimesulide, Orgotein, Oxaceprol, Oxazole Derivatives, Paranyline, Pifoxime, 2-substituted-4,6-di-tertiary-butyl-s-hydroxy-1,3-pyrimidines, Proquazone and Tenidap.

g. Antiseptics
Guanidines such as Alexidine, Ambazone, Chlorhexidine and Picloxydine;
Halogens/Halogen Compounds such as Bomyl Chloride, Calcium Iodate, Iodine, Iodine Monochloride, Iodine Trichloride, Iodoform, Povidone-Iodine, Sodium Hypochlorite, Sodium Iodate, Symclosene, Thymol Iodide, Triclocarban, Triclosan and Troclosene Potassium;
Nitrofurans such as Furazolidone, 2-(Methoxymethyl)-5-Nitrofuran, Nidroxyzone, Nifuroxime, Nifurzide and Nitrofurazone;
Phenols such as Acetomeroctol, Chloroxylenol, Hexachlorophene, 1-Naphthyl Salicylate, 2,4,6-Tribromo-m-cresol and 3′,4′,5-Trichlorosalicylanilide;
Quinolines such as Aminoquinuride, Chloroxine, Chlorquinaldol, Cloxyquin, Ethylhydrocupreine, Halquinol, Hydrastine, 8-Hydroxyquinoline and Sulfate; and
others, such as Boric Acid, Chloroazodin, m-Cresyl Acetate, Cupric sulfate, and Ichthammol.

h. Antivirals
Purines/Pyrimidinones, such as 2-Acetyl-Pyridine 5-((2-pyridylamino)thiocarbonyl) Thiocarbonohydrazone, Acyclovir, Dideoxyadenosine, Dideoxycytidine, Dideoxyinosine, Edoxudine, Floxuridine, Ganciclovir, Idoxuridine, MADU, Pyridinone, Trifluridine, Vidrarbine and Zidovudline;
others such as Acetylleucine Monoethanolamine, Acridinamine, Alkylisooxazoles, Amantadine, Amidinomycin, Cuminaldehyde Thiosemicarbzone, Foscamet Sodium, Kethoxal, Lysozyme, Methisazone, Moroxydine, Podophyllotoxin, Ribavirin, Rimantadine, Stallimycin, Statolon, Thymosins, Tromantadine and Xenazoic Acid.

In certain embodiments, the invention is directed to methods of binding opioid receptors, particularly δ opioid receptors, in a patient in need thereof, comprising the step of administering to said patient an effective amount of a compound of formula I. The δ opioid receptors may be located in the central nervous system or located peripherally to the central nervous system. In certain preferred embodiments, the binding of the ligand modulates the activity, preferably as an agonist, of said opioid receptors. In certain preferred embodiments, the compound of formula I does not substantially cross the blood-brain barrier. Preferably, the compounds of the present invention are peripherally selective.

In certain embodiments of the method, the patient is in need of prevention or treatment of a condition or disease caused by an opioid, either endogenous or exogenous.

The carboxamide and amino derivatives of the present invention and pharmaceutical compositions containing these compounds may be utilized in a number of ways. In certain embodiments, the carboxamide derivatives are ligands of the δ opioid receptor and are useful, inter alia, in methods for treating and/or preventing pain, gastrointestinal disorders, ileus, urogenital tract disorders, immunomodulatory disorders, inflammatory disorders, respiratory function disorders, anxiety, mood disorders, stress-related disorders, sympathetic nervous system disorder, tussis, motor disorder, traumatic injury, stroke, cardiac arrhythmia, glaucoma, sexual dysfunction, shock, brain edema, cerebral ischemia, cerebral deficits subsequent to cardiac bypass surgery and grafting, systemic lupus erythematosus, Hodgkin's disease, Sjogren's disease, epilepsy, and rejection in organ transplants and skin grafts, and substance addiction. In certain other embodiments, the carboxamide derivatives are ligands of the δ opioid receptor and are useful, inter alia, in methods for providing cardioprotection following myocardial infarction, in methods for providing and maintaining an anesthetic state, and in methods of detecting, imaging or monitoring degeneration or dysfunction of opioid receptors in a patient.

In certain embodiments, the invention is directed to methods for preventing or treating pain, comprising the step of administering to a patient in need thereof an effective amount of a compound of formula I. The method may further comprise the step of administering to said patient an effective amount of an opioid.

Suitable opioids include alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, diastereoisomers thereof, pharmaceutically acceptable salts, thereof, complexes thereof and mixtures thereof.

In certain embodiments, the invention is directed to methods for preventing or treating gastrointestinal dysfunction, including ileus, comprising the step of administering to a patient in need thereof an effective amount of a compound of formula I.

In certain embodiments, the invention is directed to methods for preventing or treating urogenital tract disorder, including incontinence, comprising the step of administering to a patient in need thereof an effective amount of a compound of formula I.

In certain embodiments, the invention is directed to methods for preventing or treating immunomodulatory disorder, comprising the step of administering to a patient in need thereof an effective amount of a compound of formula I. Immunomodulatory disorders include, but are not limited to, autoimmune disease, collagen disease, allergies, side effects associated with the administration of an anti-tumor agent, and side effects associated with the administration of an antiviral agent. Autoimmune diseases include, but are not limited to, arthritis, an autoimmune disorder associated with a skin graft, an autoimmune disorder associated with organ transplant, and an autoimmune disorder associated with surgery.

In certain embodiments, the invention is directed to methods for preventing or treating inflammatory disorder, comprising the step of administering to a patient in need thereof an effective amount of a compound of formula I. Inflammatory disorders include, but are not limited to, arthritis, psoriasis, asthma, or inflammatory bowel disease.

In certain embodiments, the invention is directed to methods for preventing or treating respiratory function disorder, comprising the step of administering to a patient in need thereof an effective amount of a compound of formula I.

In certain embodiments, the invention is directed to methods for preventing or treating anxiety, mood disorders, and/or stress-related disorders, comprising the step of administering to a patient in need of such treatment an effective amount of a compound of formula I. Stress-related disorders include, but are not limited to, post-traumatic stress disorder, panic disorder, generalized anxiety disorder, social phobia, and obsessive compulsive disorder.

In certain embodiments, the invention is directed to methods for preventing or treating sympathetic nervous system disorders, including hypertension, comprising the step of administering to a patient in need of such treatment an effective amount of a compound of formula I.

In certain embodiments, the invention is directed to methods for preventing or treating tussis, comprising the step of administering to a patient in need of such treatment an effective amount of a compound of formula I.

In certain embodiments, the invention is directed to methods for preventing or treating motor disorders, including tremors, Parkinson's disease and Tourette syndrome, comprising the step of administering to a patient in need of such treatment an effective amount of a compound of formula I.

In certain embodiments, the invention is directed to methods for preventing or treating traumatic injury is traumatic injury to the central nervous system, including the spinal cord or brain, comprising the step of administering to a patient in need of such treatment an effective amount of a compound of formula I.

In certain embodiments, the invention is directed to methods for preventing or treating stroke, comprising the step of administering to a patient in need of such treatment an effective amount of a compound of formula I.

In certain embodiments, the invention is directed to methods for providing cardioprotection following myocardial infarction, comprising the step of administering to a patient in need of such treatment an effective amount of a compound of formula I.

In certain embodiments, the invention is directed to methods for preventing or treating substance addiction, including alcohol addiction, nicotine addiction, and drug addiction such opioid addiction, comprising the step of administering to a patient in need of such treatment an effective amount of a compound of formula I.

In certain embodiments, the invention is directed to methods for producing or maintaining an anesthetic state, comprising the step of administering to a patient in need of such treatment an effective amount of a compound of formula I. The method may further comprise the step of administering to said patient an anesthetic agent, preferably co-administered with the compound of formula I, selected from the group consisting of an inhaled anesthetic, a hypnotic, an anxiolytic, a neuromuscular blocker and an opioid. Compounds of the invention are useful as analgesic agent for use during general anesthesia and monitored anesthesia care. Combinations of agents with different properties may be used to achieve a balance of effects needed to maintain the anesthetic state.

In certain aspects, the invention is directed to the isotopically- and radio-labeled derivatives of carboxamide derivative of formula I. Suitable labeled derivatives include ²H, ³H, ¹¹C, ¹³C, ¹³N, ¹⁵N, ¹⁵O, ¹⁸O, ¹⁸F and ³⁴S Such labeled derivatives are useful for biological studies, for example, using positron emission tomography, for metabolite identification studies and the like. The isotopically- and radio-labeled derivatives may be prepared by techniques well known in the art.

The present invention will now be illustrated by reference to the following specific, non-limiting examples. Those skilled in the art of organic synthesis may be aware of still other synthetic routes to the invention compounds. The reagents and intermediates used herein are either commercially available or prepared according to standard literature procedures.

Chemical Synthesis

All of the compounds were synthesized by classical methods in solution or on solid support, the latter using commercially available Rink or Wang resin and Fmoc chemistry. Amide coupling was mediated by DIC-HOBt or by HATU. The strategy of minimal side-chain protection was adopted. 9-Fluorenylmethoxycarbonyl (Fmoc) protection was used throughout for the α-amino group protection and the tert-butyloxycarbonyl (Boc) protection was used for the α-amino group protection during the last coupling step with phenylalanine(4-carboxamide) derivative. When tyrosine was used, the phenolic group was unprotected. Arginine group was introduced with 2,2,5,7,8-pentamethylchroman-6-sulfonyl (Pmc) protection, and lysine group with tert-butyloxycarbonyl (Boc) protection. Protecting groups were removed up on resin cleave with hydrofluoric acid or trifluoroacetic acid.

All amino acids were purchased from Neosystem, Novabiochem, Advanced Chemtech or Aldrich Co. (S)-Boc-NH-Cpa-OH was purchased from RSP Amino Acid Analogues Inc. General laboratory solvents were obtained from Aldrich Co. and Mallinckrodt Baker Inc. PS-Wang and PS-Rink-NH-Fmoc Resin (˜1.20 mmol/g) were purchased from Argonaut Technologies Inc. NMR analyses were performed on a Brucker AC400. All NMR spectra were recorded in CD₃OD, and calibrated against the methanol signal (3.34 ppm). LC/MS analyses were performed on a Finnigan AQA Thermo Quest and on Alliance Waters 2695. Preparative HPLC were performed on a Gilson 220.

Abbreviations:

Boc=tert-butyloxycarbonyl; DCM=dichloromethane; DIC=N,N-diisopropylcarbodiimide; DIEA=diisopropylethylamine; DMF=dimethylformamide; DMAP=4-dimethylaminopyridine; Fmoc=9-Fluorenylmethoxycarbonyl; HATU=O-(7-azabenzotriazol-1-yl)-N,N,N′,N′-tetramethyluronium hexafluorophosphate; HOBt=hydroxybenzotriazole; MeOH=methanol; Pmc=2,2,5,7,8-pentamethylchroman-6-sulfonyl; Cpa=(S)-2-Amino-3-(4-carboxamidophenyl)propionic acid; TFA=trifluoroacetic acid; TIS=triisopropylsilane; dab=(2R)-2,4-diaminobutanoic acid; Tic=(L)-1,2,3,4-tetrahydroisoquinoline-3-carboxylic acid; Leu=L-leucine; leu=D-leucine; Ala=L-alanine; ala=D-alanine; Val=L-valine; val=D-valine; Ile=L-isoleucine; ile=D-isoleucine; Pro=L-proline; pro=D-proline; Met=L-methionine; met=D-methionine; Phe=L-phenylalanine; phe=D-phenylalanine; Trp=L-tryptophan; trp=D-tryptophan; Gly=glycine; Ser=L-serine; ser=D=serine; Thr=L-threonine; thr=D-threonine; Cys=L-cysteine; cys=D-cysteine; Tyr=L-tyrosine; try=D-tyrosine; Asn=L-asparagine; asn=D-asparagine; Gln=L-glutamine; gln=D-glutamine; Asp=L-aspartic acid; asp=D-aspartic acid; Glu=L-glutamic acid; glu=D-glutamic acid; Lys=L-lysine; lys=D-lysine; Arg=L-arginine; arg=D-arginine; His=L-histidine; his=D-histidine; N(CH₃)Phe=N-methyl-L-phenylalanine, Pen=L-penicillamine, pen=D-penicillamine.

General Fmoc Deprotection Procedure:

Rink resin (1 g, 1.20 mmol) or polymer supported peptides (1 g, 1.20-0.80 mmol) was treated with 15 mL of piperidine (25% vol. in DMF) for 15 minutes at room temperature. The resin was washed with DMF (5×). The piperidine treatment was repeated. Then the resin was washed with DMF (5×), Et₂O (5×) and DCM (5×), and dried in vacuum.

General Amino Acid Coupling Procedure (Method I):

Rink resin (1 g, 1.20 mmol) or polymer supported peptides (1 g, 1.20-0.80 mmol) was swelled in DCM (10 mL). Fmoc protected amino acid (3 eq.) was added to the suspension, followed sequentially by DMF (20 mL), DIC (3.5 eq.), HOBt (1 eq.) and DMAP (1 eq.). The reaction mixture was shaken for 3 hours on an orbital shaker. The resin was washed with DMF (5×). The coupling was repeated under the same conditions until the ninhydrin test was negative. Then the resin was washed with DMF (5×), Et₂O (5×) and DCM (5×), and dried in vacuum.

General Coupling Procedure for Final Coupling (Cpa Coupling) (Method II):

Resin (300 mg, 0.91-0.85 mmol/g) was swelled in DCM (5 mL). Then, Boc protected Cpa (3 eq.) was added to the suspension, followed sequentially by DMF (10 mL), HATU (4 eq.), and DIEA (5 eq.). The reaction mixture was shaken 3 hours on an orbital shaker. The resin was washed with DMF (5×). The coupling was repeated under the same conditions until the ninhydrin test was negative. Then the resin was washed with DMF (5×), Et₂O (5×) and DCM (5×), and then dried in vacuum.

General Coupling Procedure for the Cleavage Step and Boc Deprotection with TFA:

Polymer supported peptide (300 mg, 0.91-0.85 mmol/g) was treated with 3 mL of a mixture TFA/TIS/Water (92%/5%/3%) for 2 h at room temperature. For peptides containing methionine, the cocktail was TFA/TIS/Water/Ethanethiol (92.5%/2.5%/2.5%/2.5%). The TFA was evaporated and the product precipitated with diethyl ether. The precipitate was washed two times with diethyl ether, and then dried in vacuum.

### EXAMPLE 1

### H-Cpa-Gly-Gly-Phe-Leu-NH₂

Rink resin was sequentially derivatized with (L)-Fmoc-Leucine, (L)-Fmoc-Phenylalanine, Fmoc-Glycine, Fmoc-Glycine, and (S)-Boc-Phenylalanine(4-carboxamide) and cleaved according to the general procedure (Scheme 1). LCMS MH⁺=583.

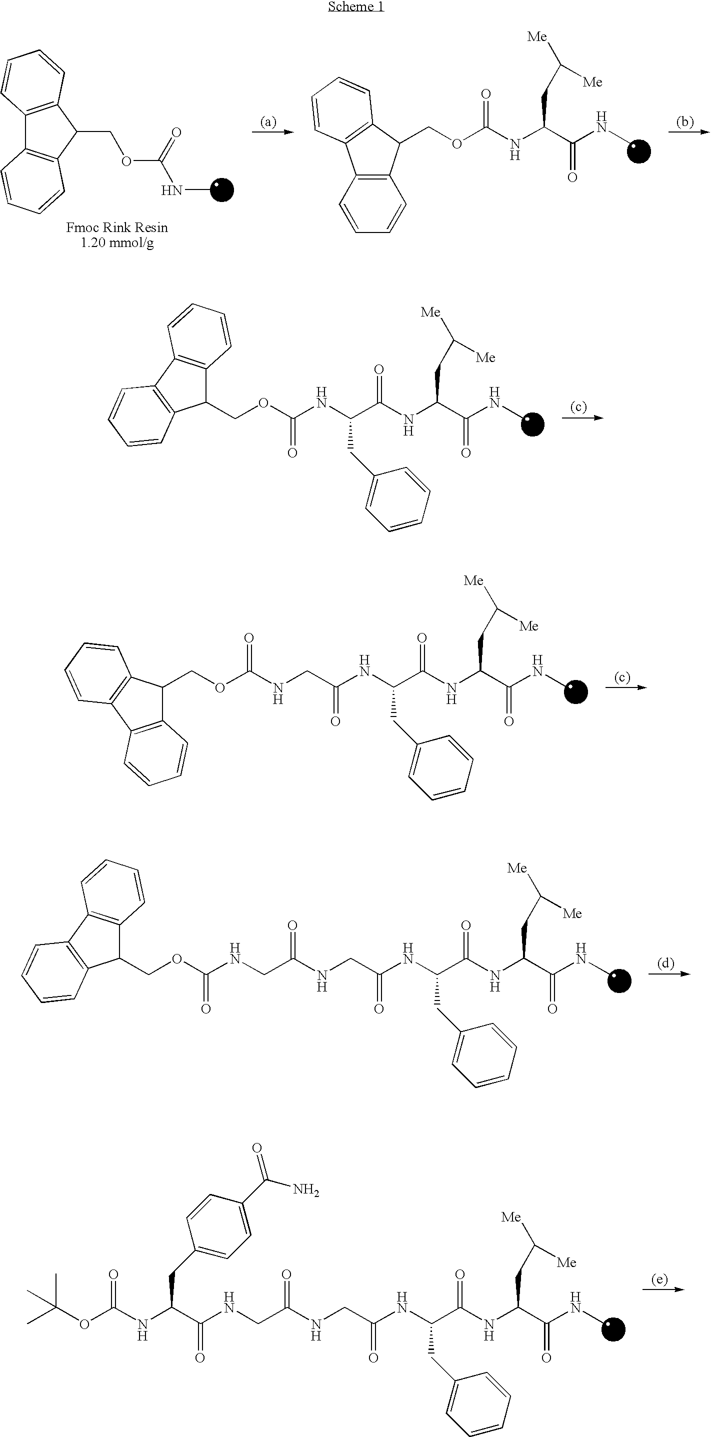

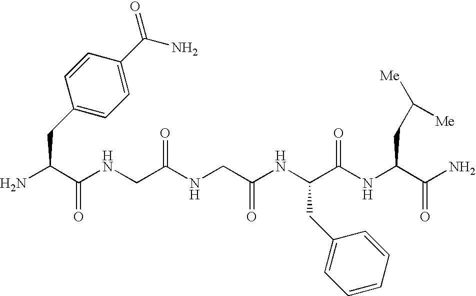

(a) (i) piperidine/DCM (25% vol.), R.T., 0.25 h., 2 times, (ii) FMOC-NH-Leu (3.0 eq), DIC (3.5 eq.), HOBt (1.0 eq.), DMAP (1.0 eq), DCM/DMF (1/2), R.T., 3.0 h., 2 times; (b) (i) piperidine/DCM (25% vol.), R.T., 0.25 h., 2 times, (ii) FMOC-NH-Phe (3.0 eq), DIC (3.5 eq.), HOBt (1.0 eq.), DMAP (1.0 eq), DCM/DMF (1/2), R.T., 3.0 h., 2 times; (c) (i) piperidine/DCM (25% vol.), R.T., 0.25 h., 2 times, (ii) FMOC-NH-Gly (3.0 eq), DIC (3.5 eq.), HOBt (1.0 eq.), DMAP (1.0 eq), DCM/DMF (1/2), R.T., 3.0 h., 2 times; (d) (i) piperidine/DCM (25% vol.), R.T., 0.25 h., 2 times, (ii) Boc-NH-Cpa (3.0 eq), HATU (4.0 eq.), DIEA (5.0 eq.), DCM/DMF (1/2), R.T., 3.0 h.; (e) TFA/TIS/Water (92/5/3), R.T., 2 h. for peptide containing methionine, TFA/TIS/Water/Ethanethiol (92/2.5/2.5/2.5).

### EXAMPLE 2

### H-Cpa-Gly-Gly-Phe-Leu

Wang resin was sequentially derivatized with (L)-Fmoc-Leucine, (L)-Fmoc-Phenylalanine, Fmoc-Glycine, Fmoc-Glycine, and (S)-Boc-Phenylalanine(4-carboxamide) (Cpa) and cleaved according to the general procedure. ¹H NMR (CD₃OD) δ(ppm)=8.58 (d, 8.9 Hz, 0.2H), 7.91 (d, 8.9 Hz, 0.1H), 7.35-7.26 (m, 4H), 7.26-7.19 (m, 1H), 7.14 (d, 8.9 Hz, 2H), 6.82 (d, 8.9 Hz, 2H), 4.73 (dd, 10.7 Hz 15.3 Hz, 1H), 4.43 (t, 8.0 Hz, 1H), 4.04-3.71 (m, 4H), 4.10 (dd, 9.1 Hz/8.0 Hz, 1H), 3.25-3.33 (m, 2H), 3.05-2.94 (m, 2H), 1.81-1.62 (m, 3H), 0.98 (d, 5.7 Hz, 3H), 0.92 (d, 5.7 Hz, 3H); MH⁺=556; retention time: _{Finnigan}=1.31 minutes; retention time: _{50° C. Waters}=8.29 minutes; purity=99.4%; Elemental analysis: H-Tyr-Gly-Gly-Phe-Leu.TFA.2H₂O: Calculated: C=51.06%; H=6.00%; N=9.92%; 0=24.94%; F=8.08% Found: C=51.10%; H=5.75%; N=9.85%.

### EXAMPLE 3

### H-Cpa-ala-Gly-Phe-leu

Wang resin was sequentially derivatized with (D)-Fmoc-Leucine, (L)-Fmoc-Phenylalanine, Fmoc-Glycine, (D)-Alanine, and (S)-Boc-Phenylalanine(4-carboxamide) (Cpa) and cleaved according to the general procedure. LCMS MH⁺=598.

### EXAMPLE 4

### H-Cpa-ala-Gly-Phe-leu-NH₂

Rink resin was sequentially derivatized with (D)-Fmoc-Leucine, (L)-Fmoc-Phenylalanine, Fmoc-Glycine, (D)-Fmoc-Alanine, and (S)-Boc-Phenylalanine(4-carboxamide) (Cpa) and cleaved according to the general procedure. ¹H NMR (CD₃OD) δ(ppm)=8.84 (d, 4.8 Hz, 0.3H), 8.18 (d, 7.3 Hz, 0.7H), 7.93 (d, 7.3 Hz, 2H), 7.43 (d, 7.3 Hz, 2H), 7.37-7.16 (m, 5H), 4.64-4.54 (m, 1H), 4.30-4.12 (m, 3H), 3.94-3.74 (m, 2H), 3.28-3.14 (m, 2H), 3.14-3.00 (m, 2H), 1.63-1.43 (m, 2H), 1.28 (d, 6.1 Hz, 3H), 1.25-1.05 (m, 1H), 0.84 (d, 6.1 Hz, 3H), 0.77 (d, 6.1 Hz, 3H); MH⁺=596; retention time: _{Finnigan}=1.43 minutes; retention time: _{50° C. Waters}=8.18 minutes; Purity=99.6%; Elemental analysis: H-Cpa-ala-Gly-Phe-leu-NH₂.2 TFA.H₂O: Calculated: C=48.51%; H=5.39%; N=11.65%; 0=21.91%; F=13.54%; Found: C=48.14%; H=5.61%; N=12.02%.

### EXAMPLE 5

### H-Cpa-Gly-Gly-Phe-leu-NH₂

Rink resin was sequentially derivatized with (D)-Fmoc-Leucine, (L)-Fmoc-Phenylalanine, Fmoc-Glycine, Fmoc-Glycine, and (S)-Boc-Phenylalanine(4-carboxamide) (Cpa) and cleaved according to the general procedure. LCMS MH⁺=583.

### EXAMPLE 6

### H-Cpa-Arg-Phe-Lys-NH₂

Rink resin was sequentially derivatized with (L)-Fmoc-ε-Boc-Lysine, (L)-Fmoc-Phenylalanine, (L)-Fmoc-Arginine(Mtr), and (S)-Boc-Phenylalanine(4-carboxamide) (Cpa) and cleaved according to the general procedure. ¹H NMR (CD₃OD) δ(ppm)=8.38 (d, 7.4 Hz, 0.8H), 8.24 (d, 7.4 Hz, 0.8H), 7.86 (d, 7.4 Hz, 2H), 7.42-7.28 (m, 6H), 7.27-7.19 (m, 1H), 4.67-4.59 (m, 1H), 4.41 (t, 6.7 Hz, 1H), 4.33-4.26 (m, 1H), 4.20 (t, 6.7 Hz, 1H), 3.28-3.15 (m, 4H), 3.12-3.00 (m, 2H), 2.95 (t, 6.7 Hz, 2H), 1.98-1.55 (m, 8H), 1.53-1.36 (m, 2H); MH⁺=639; R.T. _{Finnigan}=0.24 minutes; R.T. _{50° C. Waters}=4.87 minutes; Purity=98.2%.

### EXAMPLE 7

### H-Cpa-arg-Phe-Lys-NH₂

Rink resin was sequentially derivatized with (L)-Fmoc-ε-Boc-Lysine, (L)-Fmoc-Phenylalanine, (D)-Fmoc-Arginine(Mtr), and (S)-Boc-Phenylalanine(4-carboxamide) (Cpa) and cleaved according to the general procedure. Elemental analysis: NH₂-Cpa-arg-Phe-Lys-NH₂.5 TFA.3H₂O. Calculated: C=38.99%; H=4.55%; N=11.09%; 0=20.84%; F=22.56%. Found: C=38.63%; H=4.50%; N=11.32%.

### EXAMPLE 8

### H-Cpa-Pro-Trp-Phe-NH₂

Rink resin was sequentially derivatized with (L)-Fmoc-Phenylalanine, (L)-Fmoc-Tryptophan, (L)-Fmoc-Proline, and (S)-Boc-Phenylalanine(4-carboxamide) (Cpa) and cleaved according to the general procedure. ¹H NMR (CD₃OD) δ(ppm)=7.97 (d, 7.1 Hz, 0.3H), 7.90-7.91 (m, 2H), 7.68-7.63 (m, 1H), 7.45-7.35 (m, 2H), 7.34-7.17 (m, 6H), 7.17-7.04 (m, 3H), 4.76-4.53 (m, 2H), 4.49 (dd, 5.3 Hz/8.0 Hz, 1H), 4.40 (t, 6.7 Hz, 1H), 3.67-3.56 (m, 1H), 3.44-3.22 (m, 3H), 3.19-3.04 (m, 2H), 3.01-2.87 (m, 2H), 2.10-1.99 (m, 1H), 1.93-1.74 (m, 2H) 1.61-1.202 (m, 1H); MH⁺=638; R.T. _{Finnigan}=1.41 minutes; R.T. _{5° C. Waters}=8.31 minutes; Purity=95.5%.

### EXAMPLE 9

### H-Cpa-ala-Phe-Gly-Tyr-Pro-Ser

Rink resin was sequentially derivatized with (L)-Fmoc-OtBu-Serine, (L)-Fmoc-Proline, (L)-Fmoc-Tyrosine, and (L)-Fmoc-Phenylalanine, (D)-Fmoc-Alanine, and (S)-Boc-Phenylalanine(4-carboxamide) (Cpa) and cleaved according to the general procedure. ¹H NMR (CD₃OD) δ(ppm)=8.60 (d, 8.3 Hz, 0.3H), 8.50 (d, 8.3 Hz, 0.5H), 8.35 (d, 8.3 Hz, 0.3H), 8.31 (t, 6.2 Hz, 0.5H), 8.21 (d, 8.3 Hz, 0.3H), 8.17 (t, 6.2 Hz, 0.3H), 8.11 (d, 8.3 Hz, 0.4H), 7.98 (d, 8.3 Hz, 0.4H), 7.90 (dd, 8.3 Hz/1.0 Hz, 2H), 7.38 (t, 6.2 Hz, 2H), 7.35-7.19 (m, 5H), 7.11 (dd, 16.5 Hz/8.3 Hz, 2H), 6.77 (dd, 10.3 Hz/8.3 Hz, 2H), 4.72-4.54 (m, 1H), 4.54-4.46 (m, 1H), 4.44-4.36 (m, 1H), 4.25-4.10 (m, 2H), 4.04-3.62 (m, 6H), 3.59-3.45 (m, 1H), 3.30-3.04 (m, 3H), 3.00-2.80 (m, 2H), 2.27-2.15 (m, 1H), 2.13-1.91 (m, 3H), 1.82-1.65 (m, 1H), 1.00 (d, 8.0 Hz, 3H); MH⁺=831; R.T. _{Finnigan}=1.29 minutes; R.T. _{50° C. Waters}=7.20 minutes; Purity=98.0%.

### EXAMPLE 10

### H-Cpa-met-Phe-His-Leu-Met-Asp

Rink resin was sequentially derivatized with (L)-Fmoc-OtBu-Aspartic acid, (L)-Fmoc-Methionine, (L)-Fmoc-N(Boc)Histidine, and (L)-Fmoc-Phenylalanine, (D)-Fmoc-Methionine, and (S)-Boc-Phenylalanine(4-carboxamide) (Cpa) and cleaved according to the general procedure. ¹H NMR (CD₃OD) δ(ppm)=8.87 (s, 1H), 7.92 (d, 8.2 Hz, 2H), 7.42 (s, 1H), 7.40 (d, 8.2 Hz, 2H), 7.35-7.20 (m, 5H), 4.81-4.71 (m, 2H), 4.66 (dd, 10.6 Hz/4.7 Hz, 1H), 4.56 (dd, 8.2 Hz/4.7 Hz, 1H), 4.35 (dd, 9.4 Hz/5.9 Hz, 1H), 4.23 (dd, 8.2 Hz/5.9 Hz, 1H), 4.16 (t, 8.2 Hz, 1H), 3.28-3.15 (m, 5H), 2.90 (d, 6.7 Hz, 2H), 2.82 (dd, 14.4 Hz/11.1 Hz, 1H), 2.67-2.53 (m, 2H), 2.13 (s, 3H), 2.04-1.82 (m, 3H), 1.95 (s, 3H), 1.80-1.46 (m, 6H), 1.02 (d, 6.1 Hz, 3H), 0.87 (d, 6.1 Hz, 3H); MH⁺=983; R.T. _{Finnigan}=1.59 minutes; R.T. _{50° C. Waters}=9.03 minutes; Purity=95.4%.

### EXAMPLE 11

### H-Cpa-ser-Gly-Phe-Leu-Thr

Wang resin was sequentially derivatized with (L)-Fmoc-OtBu-Threonine, (L)-Fmoc-Leucine, (L)-Fmoc-Phenylalanine, Fmoc-Glycine, (D)-Fmoc-Serine, and (S)-Boc-Phenylalanine(4-carboxamide) (Cpa) and cleaved according to the general procedure. ¹H. NMR (CD₃OD) δ(ppm)=8.67 (d, 7.3 Hz, 0.7H), 7.92 (d, 7.3 Hz, 2H), 7.75 (d, 7.3 Hz, 2H), 7.46 (d, 7.3 Hz, 2H), 7.36-7.27 (m, 4H), 7.26-7.19 (m, 1H), 4.80-4.70 (m, 1H), 4.45-4.40 (m, 2H), 4.40-4.24 (m, 3H), 3.90 (d, 13.3 Hz, 1H), 3.82-3.63 (m, 3H), 3.24-3.11 (m, 3H), 3.02 (dd, 11.1 Hz/8.9 Hz, 1H), 1.78-1.61 (m, 3H), 1.20 (d, 6.7 Hz, 3H), 1.01 (d, 6.7 Hz, 3H), 0.94 (d, 6.7 Hz, 3H); MH⁺=714; R.T. _{Finnigan}=1.23 minutes; R.T. _{50° C. Waters}=7.00 minutes; Purity=99.0%.

### EXAMPLE 12

### H-Cpa-Gly-Gly-Phe-Leu-Arg-Arg-Ile-Arg-Pro-Lys

Wang resin was sequentially derivatized with (L)-Fmoc-ε-Boc-Lysine, (L)-Fmoc-Proline, (L)-Fmoc-Arginine(Pmc), (L-Fmoc-Isoleucine, (L-Fmoc-Arginine(Pmc), (L-Fmoc-Arginine(Pmc), (L-Fmoc-Leucine, (L)Fmoc-Phenylalanine, Fmoc-Glycine, Fmoc-Glycine, and (S)-Boc-Phenylalanine(4-carboxamide) (Cpa) and cleaved according to the general procedure. Elemental analysis: NH₂-Cpa-Gly-Gly-Phe-Leu-Arg-Arg-Ile-Arg-Pro-Lys-OH.6 TFA.2H₂O. Calculated: C=43.27%; H=5.45%; N=14.61%; 0=20.47%; F=16.21%. Found: C=43.54%; H=5.63%; N=14.28%. ¹H NMR (CD₃OD) 8 (ppm)=8.46 (d, 8.2 Hz, 0.6H), 8.30-8.16 (m, 2H), 8.09 (d, 7.3 Hz, 0.6H), 8.01 (d, 7.3 Hz, 0.8H), 7.92 (d, 8.2 Hz, 2H), 7.45 (d, 8.2 Hz, 2H), 7.36-7.22 (m, 5H), 4.71-4.61 (m, 1H), 4.58-4.46 (m, 2H), 4.43-4.19 (m, 6H), 4.05-3.76 (m, 5H), 3.73-3.63 (m, 1H), 3.30-3.14 (m, 9H), 3.06 (dd, 14.5 Hz/8.2 Hz, 1H), 2.99 (t, 7.3 Hz, 2H), 2.35-2.23 (m, 1H), 2.12-1.46 (m, 25H), 1.03-0.84 (m, 12H); MH⁺=1390; retention time: Finnigan=1.16 minutes; retention time: _{50° C. Waters}=7.03 minutes; purity=95.0%.

### EXAMPLE 13

### H-Cpa-ala-Gly-N(Me)Phe-NHCH₂CH₂OH

This material was prepared in identical fashion as described by the method reported in Neugebauer, Witold; Escher, Emanuel; Helvetica Chimica Acta 1989, 72, 1319-23, except that (S)-Boc-phenylalanine(4-carboxamide) (Cpa) was used in place of Boc-tyrosine. LCMS MH⁺=542.

### EXAMPLE 14

### Cpa-Tic-Imidazole

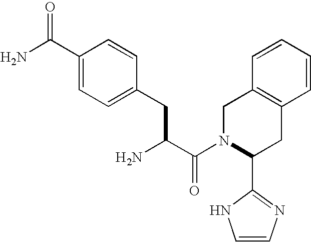

Imidazole A (2 mmol; Pharmacore Inc.) was coupled with (S)-Boc-phenylalanine(4-carboxamide) (2.1 mmol; Cpa) and deprotected using the general procedure: 87% yield. LCMS MH+390.

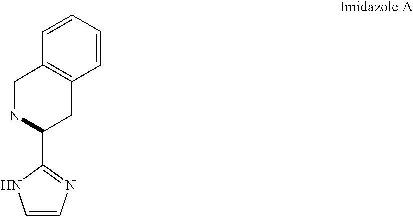

### EXAMPLE 15

### Cpa-c[dab-Gly-Phe-Leu]

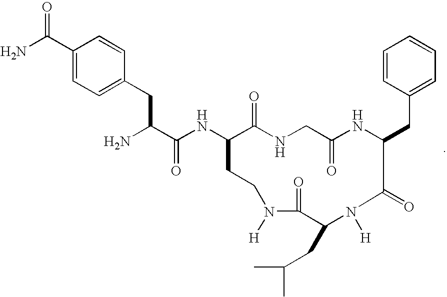

This material is prepared in identical fashion as described in DiMaio, J.; Lemieux, C.; Schiller, P. W., Life Sciences, 1982, 31, 2253-2256, except that (S)-phenylalanine(4-carboxamide) is used in place of (L)-tyrosine.

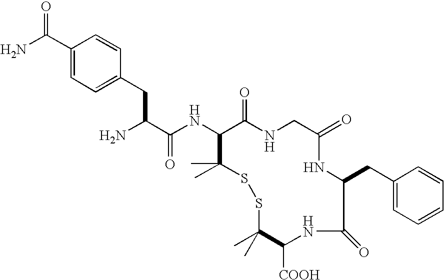

### EXAMPLE 16

### Cpa-c[pen-Gly-Phe-pen]

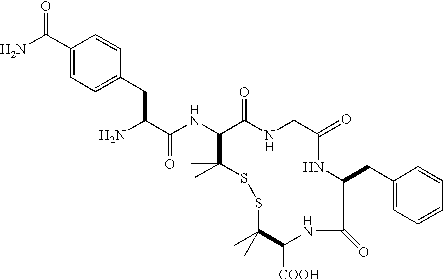

This material is prepared in identical fashion as described in Mosberg, H. I., et al., Proc. Natl. Acad. Science USA, 1983, 80, 5871-5874, except that (S)-phenylalanine(4-carboxamide) is used in place of (L)-tyrosine.

### EXAMPLE 17

### Cpa-ctpen-Gly-Phe-cys]-OH

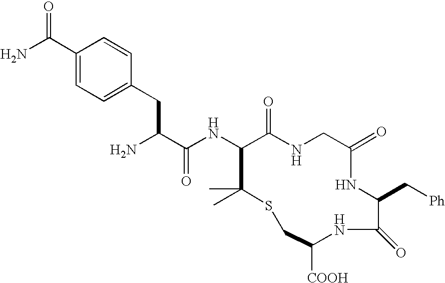

This material is prepared in identical fashion as described in Baker, T. J., et al., Pure Applied Chemistry, 2000, 72, 347-354, except that (S)-phenylalanine(4-carboxamide) is used in place of (L)-tyrosine.

### EXAMPLE 18

### Cpa-3-[(2-aminoethyl)methylamino]-D-alanylglycyl-Phe (4 to 2)-lactam

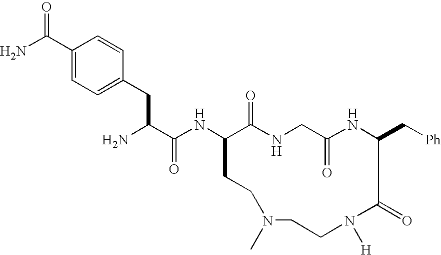

This material is prepared in identical fashion as described in Baker, T. J. et al. Pure Applied Chemistry, (2000), 72, 347-354 and J. Medicinal Chemistry (1998), 41, 2631-2635 except that (S)-Phenylalanine(4-carboxamide) is used in place of (L)-Tyrosine.

Biological Activity:

The potencies of the compounds of the Examples were determined by testing the ability of a range of concentrations of each compound to inhibit the binding of the non-selective opioid antagonist, [³H]diprenorphine, to the cloned human μ, κ, and δ opioid receptors, expressed in separate cell lines. IC₅₀ values were obtained by nonlinear analysis of the data using GraphPad Prism version 3.00 for Windows (GraphPad Software, San Diego). Kᵢ values were obtained by Cheng-Prusoff corrections of IC₅₀ values.

Receptor Binding

The receptor binding method (DeHaven and DeHaven-Hudkins, 1998) was a modification of the method of Raynor et al. (1994). After dilution in buffer A and homogenization as before, membrane proteins (10-80 μg) in 250 μL were added to mixtures containing test compound and [³H]diprenorphine (0.5 to 1.0 nM, 40,000 to 50,000 dpm) in 250 μL of buffer A in 96-well deep-well polystyrene titer plates (Beckman). After incubation at room temperature for one hour, the samples were filtered through GF/B filters that had been presoaked in a solution of 0.5% (w/v) polyethylenimine and 0.1% (w/v) bovine serum albumin in water. The filters were rinsed 4 times with 1 mL of cold 50 mM Tris HCl, pH 7.8 and radioactivity remaining on the filters determined by scintillation spectroscopy. Nonspecific binding was determined by the minimum values of the titration curves and was confirmed by separate assay wells containing 10 μM naloxone. Kᵢ values were determined by Cheng-Prusoff corrections of IC₅₀ values derived from nonlinear regression fits of 12 point titration curves using GraphPad Prism® version 3.00 for Windows (GraphPad Software, San Diego, Calif.).

To determine the equilibrium dissociation constant for the inhibitors (Kᵢ), radioligand bound (cpm) in the presence of various concentrations of test compounds was measured. The concentration to give half-maximal inhibition (EC₅₀) of radioligand binding was determined from a best nonlinear regression fit to the following equation,

$$Y = Bottom + \frac{( Top - Bottom )}{1 + {10}^{X - Log EC50}}$$

where Y is the amount of radioligand bound at each concentration of test compound, Bottom is the calculated amount of radioligand bound in the presence of an infinite concentration of test compound, Top is the calculated amount of radioligand bound in the absence of test compound, X is the logarithm of the concentration of test compound, and LogEC₅₀ is the log of the concentration of test compound where the amount of radioligand bound is half-way between Top and Bottom. The nonlinear regression fit was performed using the program Prism® (GraphPad Software, San Diego, Calif.). The Kᵢ values were then determined from the EC₅₀ values by the following equation,

$${K}_{i} = \frac{{EC}_{50}}{1 + \frac{[ ligand ]}{{K}_{d}}}$$
where [ligand] is the concentration of radioligand and K_{d} is the equilibrium dissociation constant for the radioligand.
   Receptor-Mediated [³⁵S]GTPγS binding

The potency and efficacy of compounds at each of the receptors are assessed by modifications of the methods of Selley et al., 1997 and Traynor and Nahorski, 1995 using receptor-mediated [³⁵S]GTPγS binding in the same membrane preparations used to measure receptor binding. Assays are carried out in 96-well FlashPlates® (Perkin Elmer Life Sciences, Inc, Boston, Mass.). Membranes prepared from CHO cells expressing the appropriate receptor (50-100 μg of protein) are added to assay mixtures containing agonist with or without antagonists, 100 pM [³⁵S]GTPγS (approx. 100,000 dpm), 3.0 μM GDP, 75 mM NaCl, 15 mM MgCl₂, 1.0 mM ethylene glycol-bis(β-aminoethyl ether)-N,N,N N′-tetracetic acid, 1.1 mM dithiothreitol, 10 μg/mL leupeptin, 10 μg/mL pepstatin A, 200 μg/mL bacitracin, and 0.5 μg/mL aprotinin in 50 mM Tris-HCl buffer, pH 7.8. After incubation at room temperature for one hour, the plates are sealed, centrifuged at 800×g in a swinging bucket rotor for 5 minutes and bound radioactivity determined with a TopCount microplate scintillation counter (Packard Instrument Co., Meriden, Conn.).

EC₅₀ values for agonists are determined from nonlinear regression fits of 8- or 12-point titration curves to the 4-parameter equation for a sigmoidal dose-response with a slope factor of 1.0 using GraphPad Prism® version 3.00 for Windows (GraphPad Software, San Diego, Calif.).

Results and Discussions

The potencies of the compounds were determined by testing the ability of a range of concentrations of each compound to inhibit the binding of the non-selective opioid antagonist, [³H]diprenorphine, to the cloned human μ, κ, and δ opioid receptors, expressed in separate cell lines. All the compounds tested (Examples 1 to 14) bind with high affinity to the human cloned δ opioid receptor. These compounds display high selectivity δ/κ and δ/μ. The potencies of the ligands were assessed by their abilities to stimulated [³⁵S]GTPγS binding to membranes containing the cloned human δ opioid receptors. All the compounds tested were agonists at 6 opioid receptor with EC₅₀ values in the nanomolar range. Compounds in the Examples 1 to 14 possessed a Kᵢ of less than 1 mM against the δ receptor. As a specific example, compound of Example 1 possessed a Kᵢ=7 nM and an EC₅₀=74 nM against the δ receptor with greater than 10-fold selectivity versus the μ and κ opioid receptors.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A compound of formula I: 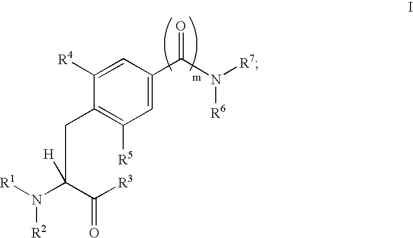
or a stereoisomer, prodrug, pharmaceutically acceptable salt, hydrate, solvate, acid salt hydrate, N-oxide or isomorphic crystalline form thereof.
wherein:
R¹ and R² are each, independently, H, alkyl, alkenyl, or —C(═NH)NH₂, provided that at least one of R¹ and R² is other than —C(═NH)NH₂, or R¹ and R² taken together with the nitrogen atom to which they are attached form a 4- to 8-membered heterocycloalkyl ring wherein the heterocycloalkyl ring is optionally interrupted with one additional O, N, or S heteroatom;
R³ is -[J]ₒ-X, 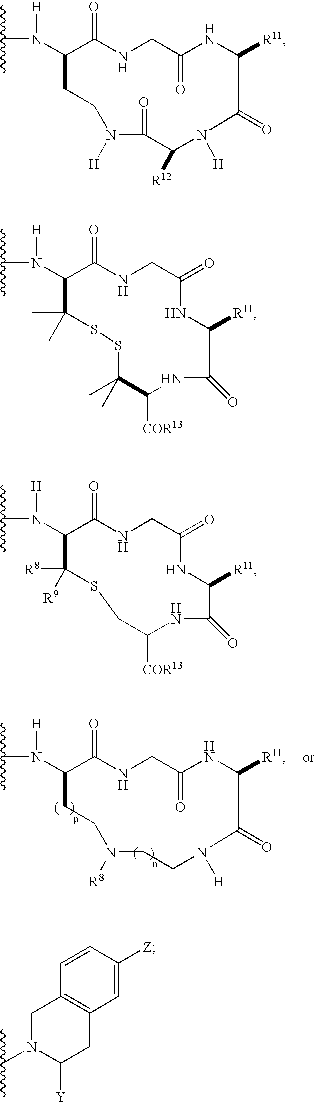
J is [NR^{8a}(C(R^{9a})(R¹⁰))ₙ—C(═O)];
X is OR⁸, NR^{8b}R^{9b}, or —N(R^{8c})CH(R^{9c})(R¹⁰);
R⁴ and R⁵ are each, independently, H or methyl;
R⁶ and R⁷ are each, independently, H or alkyl, or R⁶ and R⁷ taken together with the nitrogen atom to which they are attached form a 4- to 8-membered heterocycloalkyl ring wherein the heterocycloalkyl ring is optionally interrupted with one additional O, N, or S heteroatom;
R⁸ and R⁹ are each, independently, H or alkyl;
R^{8a} and R^{9a} are each, independently, H or alkyl, or R^{8a} and R^{9a} taken together with the nitrogen and carbon atoms through which they are connected form a 4- to 14-membered heterocycloalkyl ring wherein the heterocycloalkyl ring is optionally interrupted with one additional O, N, or S heteroatom;
R^{8b} and R^{9b} are each, independently, H or alkyl, or R^{8b} and R^{9b} taken together with the nitrogen atom to which they are attached form a 4- to 8-membered heterocycloalkyl ring wherein the heterocycloalkyl ring is optionally interrupted with one additional O, N, or S heteroatom;
R^{8c} and R^{9c} are each, independently, H or alkyl, or R^{8c} and R^{9c} taken together with the nitrogen and carbon atoms through which they are connected form a 4- to 8-membered heterocycloalkyl ring, wherein the heterocycloalkyl ring is optionally interrupted with one additional O, N, or S heteroatom;
R¹⁰ is —(C(R¹⁴)(R¹⁵))_{q}—R¹⁶;
R¹¹ and R¹² are each, independently, H or —C(R¹⁴)(R¹⁵)R¹⁰;
R¹³ is —OR¹⁴ or —NR¹⁴R¹⁵;
R¹⁴ and R¹ are each, independently, H or alkyl;
R¹⁶ is H, alkyl, cycloalkyl, heterocycloalkyl, aryl, heteroaryl, —CR¹⁴RR¹⁵OH, —CR¹⁴R¹⁵S(O)ᵣR⁹, —CR¹⁴R¹⁵COOR⁴, —CR¹⁴ R¹⁵CONHR⁴, —CR¹⁴R¹⁵NHR⁴, or —CR¹⁴R¹⁵NH(C═NH)NH₂;
Y is heteroaryl;
Z is H or —OR⁸;
m is the integer 0 or 1;
n is an integer from 1 to 4;
o is an integer from 0 to 16;
p is an integer from 0 to 3;
q is an integer from 0 to 6; and
r is the integer 0, 1 or 2.

2. A compound according to claim 1,
wherein R¹ and R² are each, independently, H or alkyl.

3. A compound according to claim 2,
wherein R¹ and R² are each H.

4. A compound according to claim 1,
wherein each J is, independently, the amino acid glycine, phenylalanine, leucine, alanine, arginine, lysine, isoleucine, proline, tryptophan, serine, methionine, threonine, tyrosine, histidine, aspartic acid, cysteine, or (N-methyl)phenylalanine.

5. A compound according to claim 4,
wherein each J is, independently, the amino acid phenylalanine, arginine, lysine, proline, or tryptophan.

6. A compound according to claim 4,
wherein each J is, independently, the amino acid glycine, phenylalanine, leucine, or alanine.

7. A compound according to claim 4,
wherein each J is, independently, the amino acid serine, glycine, phenylalanine, leucine, or threonine.

8. A compound according to claim 4,
wherein R³ is
-[arg-Phe-Lys]-X,
-[Pro-Trp-Phe]-X,
-[Gly-Gly-Phe-Leu]-X,
-[ala-Gly-Phe-Leu]-X,
-[ala-Gly-N(CH₃)Phe]-X,
-[ser-Gly-Phe-Leu-Thr]-X,
-[ala-Phe-Gly-Tyr-Pro-Ser]-X, or
-[Gly-Gly-Phe-Leu-Arg-Arg-Ile-Arg-Pro-Lys]-X.

9. A compound according to claim 1,
wherein R³ is:

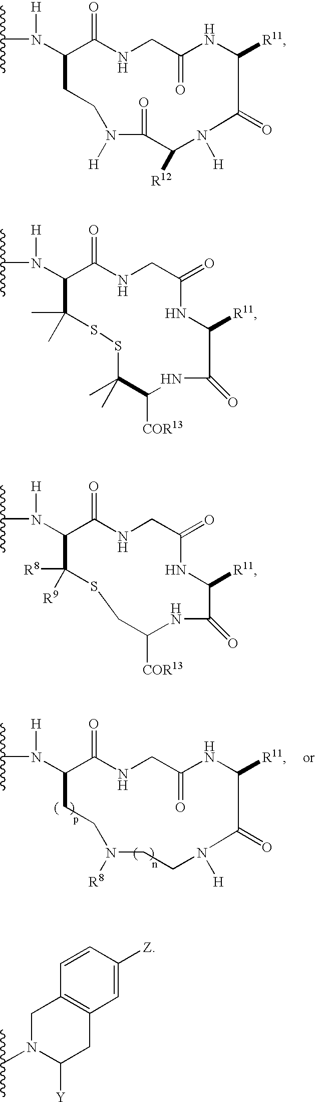

10. A compound according to claim 9,
wherein R³ is:

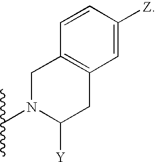

11. A compound according to claim 9,
wherein R³ is:

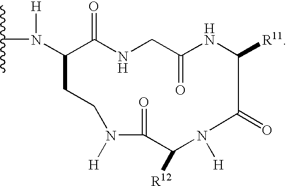

12. A compound according to claim 9,
wherein R³ is:

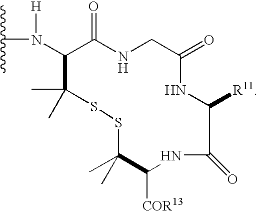

13. A compound according to claim 9,
wherein R³ is:

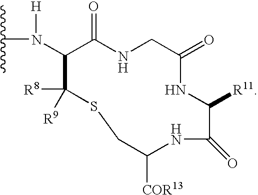

14. A compound according to claim 9,
wherein R³ is:

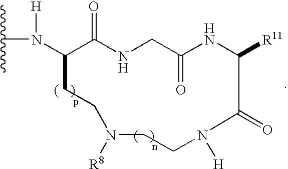

15. A compound according to claim 1,
wherein X is —OH, —NH₂, or —NHCH₂CH₂OH.

16. A compound according to claim 15,
wherein X is —OH or —NH₂.

17. A compound according to claim 16,
wherein X is —NH₂.

18. A compound according to claim 1,
wherein at least one of R⁴ and R⁵ is H.

19. A compound according to claim 1,
wherein R⁶ and R⁷ are each, independently, H or alkyl.

20. A compound according to claim 9,
wherein R⁶ and R⁷ are each H.

21. A compound according to claim 1,
wherein R¹, R², R³, R⁴, R⁵, R⁶, and R⁷ are each H.

22. A compound according to claim 1,
wherein R⁸ and R⁹ are each, independently, H or alkyl.

23. A compound according to claim 22,
wherein R⁸ and R⁹ are each H.

24. A compound according to claim 22,
wherein R⁸ and R⁹ are each methyl.

25. A compound according to claim 1,
wherein R^{8a} and R^{9a} are each, independently, H or alkyl.

26. A compound according to claim 25,
wherein R^{8a} and R^{9a} are H.

27. A compound according to claim 1,
wherein R^{8a} and R^{9a} taken together with the nitrogen and carbon atoms through which they are connected form a 4- to 14-membered heterocycloalkyl ring wherein the heterocycloalkyl ring is optionally interrupted with one additional O, N, or S heteroatom, provided that the additional heteroatom is separated by at least two carbon atoms from the nitrogen atom to which R^{8a} is attached.

28. A compound according to claim 1,
wherein R^{8b} and R^{9b} are each, independently, H or alkyl.

29. A compound according to claim 1,
wherein R^{8c} and R^{9c} are each, independently, H or alkyl.

30. A compound according to claim 1,
wherein R¹⁰ is H.

31. A compound according to claim 1,
wherein R¹¹ is aralkyl.

32. A compound according to claim 31,
wherein R¹² is benzyl.

33. A compound according to claim 1,
wherein R¹² is alkyl.

34. A compound according to claim 33,
wherein R¹² is 2-methylprop-1-yl.

35. A compound according to claim 1,
wherein R¹³ is —OH or NH₂.

36. A compound according to claim 35,
wherein R¹³ is —OH.

37. A compound according to claim 1,
wherein Y is imidazol-2-yl or oxazol-2-yl.

38. A compound according to claim 1,
wherein Z is H or —OH.

39. A compound according to claim 1,
wherein m is 1.

40. A compound according to claim 1,
wherein n is 1.

41. A compound according to claim 1,
wherein o is 0 to 10.

42. A compound according to claim 41,
wherein o is 1 to 10.

43. A compound according to claim 42,
wherein o is 1 to 6.

44. A compound according to claim 43,
wherein o is 1 to 4.

45. A compound according to claim 1,
wherein p is 1.

46. A compound according to claim 1,
wherein q is 0, 1 or 2.

47. A compound according to claim 1,
wherein r is 0.

48. A compound according to claim 1 of formula Ia:

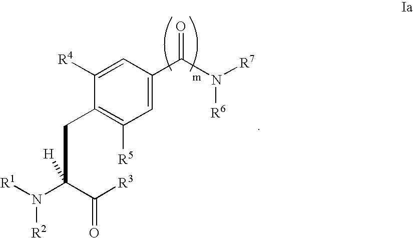

49. A compound according to claim 1, wherein R³ is:
[NR^{8d}(C(R^{9d})(R¹⁰)ₙ—C(═O)]ₛ-J-[NR^{8e}(C(R^{9e})(R¹⁰))ₙ—C(═O)]ₜ—X;
wherein:
R^{8d} and R^{9d} are each, independently, H or alkyl, or R^{8d} taken together with the R^{9d} on the carbon atom alpha to the nitrogen bearing the R^{8d} and the nitrogen and carbon atoms through which they are connected form a 4- to 14-membered heterocycloalkyl ring wherein the heterocycloalkyl ring is optionally interrupted with one additional O, N, or S heteroatom;
R^{8e} and R^{9e} are each, independently, H or alkyl, or R^{8e} taken together with the R^{9e} on the carbon atom alpha to the nitrogen bearing the R e and the nitrogen and carbon atoms through which they are connected form a 4- to 8-membered heterocycloalkyl ring wherein the heterocycloalkyl ring is optionally interrupted with one additional O, N, or S heteroatom; and
the sum of s+t is an integer of 0 to 9.

50. A compound according to claim 49,
wherein the sum of s+t is 0 to 2.

51. A compound according to claim 50,
wherein the sum of s+t is 0 or 1.

52. A compound according to claim 51,
wherein s and t are 0.

53. A compound according to claim 49,
wherein J is [NR^{8a}(CH(R^{9a})ₙ-C(═O)].

54. A compound according to claim 53,
wherein n is 1.

55. A compound according to claim 53,
wherein R^{8a} is H.

56. A compound according to claim 53,
wherein R^{9a} is H, benzyl, —(CH₂)₄—NH₂, CH₂-indol-3-yl, —(CH₂)₃—NH—C(═NH)NH₂, —CH₂CH(CH₃)₂, —CH₃, —CH₂OH, —CH(OH)CH₃, (para-hydroxyphenyl)methyl-, or —CH(CH₃)CH₂CH₃, or R^{8a} and R^{9a} taken together with the nitrogen and carbon atoms through which they are connected form a 4- to 14-membered heterocycloalkyl ring, wherein the heterocycloalkyl ring is optionally interrupted with one additional O, N, or S heteroatom.

57. A compound according to claim 56,
wherein R^{9a} is benzyl, —(CH₂)₄—NH₂, CH₂-indol-3-yl, or —(CH₂)₃—NH—C(═NH)NH₂, or R^{8a} and R^{9a} taken together with the nitrogen and carbon atoms through which they are connected form a 4- to 8-membered heterocycloalkyl ring wherein the heterocycloalkyl ring is optionally interrupted with one additional O, N, or S heteroatom.

58. A compound according to claim 1 of formula II:

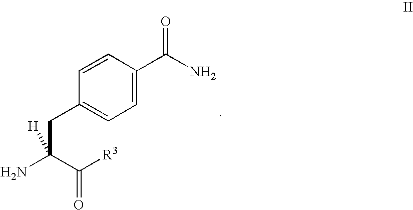

59. A compound according to claim 1, wherein said compound is:
H-(S)-2-Amino-3-(4-carboxamidophenyl)propionic acid-Gly-Gly-Phe-Leu-NH;
H-(S)-2-Amino-3-(4-carboxamidophenyl)propionic acid-Gly-Gly-Phe-Leu;
H-(S)-2-Amino-3-(4-carboxamidophenyl)propionic acid-ala-Gly-Phe-leu;
H-(S)-2-Amino-3-(4-carboxamidophenyl)propionic acid-ala-Gly-Phe-leu-NH₂;
H-(S)-2-Amino-3-(4-carboxamidophenyl)propionic acid-Gly-Gly-Phe-leu-NH₂;
H-(S)-2-Amino-3-(4-carboxamidophenyl)propionic acid-Arg-Phe-Lys-NH₂;
H-(S)-2-Amino-3-(4-carboxamidophenyl)propionic acid-arg-Phe-Lys-NH₂;
H-(S)-2-Amino-3-(4-carboxamidophenyl)propionic acid-Pro-Trp-Phe-NH₂;
H-(S)-2-Amino-3-(4-carboxamidophenyl)propionic acid-ala-Phe-Gly-Tyr-Pro-Ser;
H-(S)-2-Amino-3-(4-carboxamidophenyl)propionic acid-met-Phe-His-Leu-Met-Asp;
H-(S)-2-Amino-3-(4-carboxamidophenyl)propionic acid-ser-Gly-Phe-Leu-Thr;
H-(S)-2-Amino-3-(4-carboxamidophenyl)propionic acid-Gly-Gly-Phe-Leu-Arg-Arg-Ile-Arg-Pro-Lys;
H-(S)-2-Amino-3-(4-carboxamidophenyl)propionic acid-ala-Gly-N(Me)Phe-NHCH₂CH₂OH;
(S)-2-Amino-3-(4-carboxamidophenyl)propionic acid-c[D-A₂bu-Gly-Phe-Leu];
(S)-2-Amino-3-(4-carboxamidophenyl)propionic acid-c[D-Val_{L}-Gly-Phe-D-Ala_{L}]—OH; 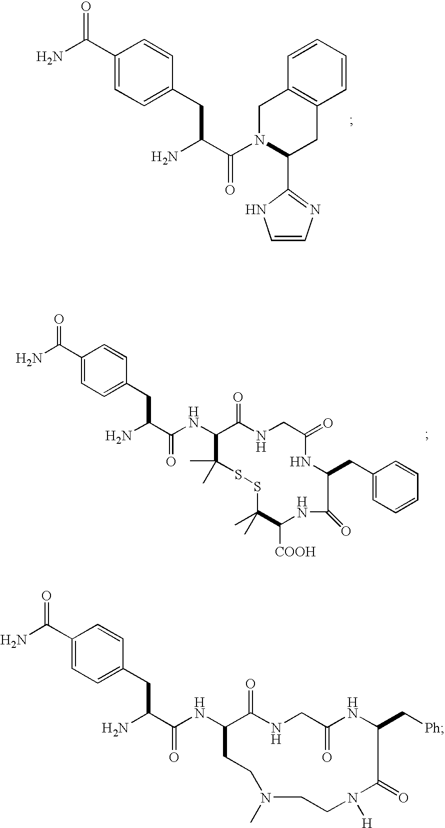
or a stereoisomer, prodrug, pharmaceutically acceptable salt, hydrate, solvate, acid salt hydrate, N-oxide or isomorphic crystalline form thereof.

60. A pharmaceutical composition, comprising:
a pharmaceutically acceptable carrier; and
an effective amount of a compound according to claim 1.

61. A pharmaceutical composition according to claim 60, further comprising:
an effective amount of at least one opioid.

62. A pharmaceutical composition according to claim 61,
wherein said opioid is selected from the group consisting of alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, diastereoisomers thereof, pharmaceutically acceptable salts thereof, complexes thereof and mixtures thereof.

63. A pharmaceutical composition according to claim 60, further comprising:
an effective amount of at least one compound selected from the group consisting of:
antibiotics, antivirals, antifungals, anti-inflammatories, anesthetics and mixtures thereof.

64. A method of binding opioid receptors in a patient in need thereof, comprising the step of:
administering to said patient an effective amount of a compound according to claim 1.

65. A method according to claim 64,
wherein said compound binds δ opioid receptors.

66. A method according to claim 65,
wherein said δ opioid receptors are located in the central nervous system.

67. A method according to claim 65,
wherein said δ opioid receptors are located peripherally to the central nervous system.

68. A method according to claim 64,
wherein said binding modulates the activity of said opioid receptors.

69. A method according to claim 68,
wherein said binding agonizes the activity of said opioid receptors.

70. A method according to claim 64,
wherein said compound does not substantially cross the blood-brain barrier.

71. A method according to claim 64,
wherein said patient is in need of prevention or treatment of a condition or disease caused by an opioid.

72. A method according to claim 71,
wherein said opioid is endogenous.

73. A method according to claim 71,
wherein said opioid is exogenous.

74. A method of preventing or treating pain, comprising the step of:
administering to a patient in need thereof an effective amount of a compound according to claim 1.

75. A method according to claim 74, further comprising the step of:
administering to said patient an effective amount of an opioid.

76. A method according to claim 75,
wherein said opioid is selected from the group consisting of alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, diastereoisomers thereof, pharmaceutically acceptable salts thereof, complexes thereof and mixtures thereof.

77. A method for preventing or treating gastrointestinal dysfunction, comprising the step of:
administering to a patient in need of such treatment an effective amount of a compound according to claim 1.

78. A method for preventing or treating ileus, comprising the step of:
administering to a patient in need of such treatment an effective amount of a compound according to claim 1.

79. A method for preventing or treating a urogenital tract disorder, comprising the step of:
administering to a patient in need of such treatment an effective amount of a compound according to claim 1.

80. A method according to claim 79,
wherein said urogenital tract disorder is incontinence.

81. A method of preventing or treating an immunomodulatory disorder, comprising the step of:
administering to a patient in need thereof an effective amount of a compound according to claim 1.

82. A method according to claim 81,
wherein said immunomodulatory disorder is selected from the group consisting of:
an autoimmune disease, a collagen disease, an allergy, a side effect associated with the administration of an anti-tumor agent, and a side effect associated with the administration of an antiviral agent.

83. A method according to claim 82,
wherein said autoimmune disease is selected from the group consisting of:
arthritis, an autoimmune disorder associated with a skin graft, an autoimmune disorder associated with organ transplant, and an autoimmune disorder associated with surgery.

84. A method for preventing or treating an inflammatory disorder, comprising the step of:
administering to a patient in need of such treatment an effective amount of a compound according to claim 1.

85. A method according to claim 84,
wherein said inflammatory disorder is arthritis, psoriasis, asthma, or inflammatory bowel disease.

86. A method for preventing or treating a respiratory function disorder, comprising the step of:
administering to a patient in need of such treatment an effective amount of a compound according to claim 1.

87. A method according to claim 86,
wherein said respiratory function disorder is asthma or lung edema.

88. A method for preventing or treating anxiety, comprising the step of:
administering to a patient in need of such treatment an effective amount of a compound according to claim 1.

89. A method for preventing or treating a mood disorder, comprising the step of:
administering to a patient in need of such treatment an effective amount of a compound according to claim 1.

90. A method according to claim 89, wherein said mood disorder is depression, bipolar manic-depression, or seasonal affective disorder.

91. A method for preventing or treating a stress-related disorder, comprising the step of:
administering to a patient in need of such treatment an effective amount of a compound according to claim 1.

92. A method according to claim 91,
wherein said stress-related disorder is post-traumatic stress disorder, panic disorder, generalized anxiety disorder, social phobia, or obsessive compulsive disorder.

93. A method for preventing or treating attention deficit hyperactivity disorder, comprising the step of:
administering to a patient in need of such treatment an effective amount of a compound according to claim 1.

94. A method for preventing or treating sympathetic nervous system disorder, comprising the step of:
administering to a patient in need of such treatment an effective amount of a compound according to claim 1.

95. A method according to claim 94,
wherein said sympathetic nervous system disorder is hypertension.

96. A method for preventing or treating tussis, comprising the step of:
administering to a patient in need of such treatment an effective amount of a compound according to claim 1.

97. A method for preventing or treating a motor disorder, comprising the step of:
administering to a patient in need of such treatment an effective amount of a compound according to claim 1.

98. A method according to claim 97,
wherein said motor disorder is tremors, Parkinson's disease, or Tourette syndrome.

99. A method for treating a traumatic injury to the central nervous system, comprising the step of:
administering to a patient in need of such treatment an effective amount of a compound according to claim 1.

100. A method according to claim 99,
wherein said traumatic injury is traumatic injury to the spinal cord or brain.

101. A method for preventing or treating stroke, comprising the step of:
administering to a patient in need of such treatment an effective amount of a compound according to claim 1.

102. A method for preventing or treating cardiac arrhythmia, comprising the step of:
administering to a patient in need of such treatment an effective amount of a compound according to claim 1.

103. A method for preventing or treating glaucoma, comprising the step of:
administering to a patient in need of such treatment an effective amount of a compound according to claim 1.

104. A method for preventing or treating sexual dysfunction, comprising the step of:
administering to a patient in need of such treatment an effective amount of a compound according to claim 1.

105. A method according to claim 104,
wherein said sexual dysfunction is premature ejaculation.

106. A method for treating a condition selected from the group consisting of shock, brain edema, cerebral ischemia, cerebral deficits subsequent to cardiac bypass surgery and grafting, systemic lupus erythematosus, Hodgkin's disease, Sjogren's disease, epilepsy, and rejection in organ transplants and skin grafts, comprising the step of:
administering to a patient in need of such treatment an effective amount of a compound according to claim 1.

107. A method for treating substance addiction, comprising the step of:
administering to a patient in need of such treatment an effective amount of a compound according to claim 1.

108. A method according to claim 107,
wherein said substance addiction is alcohol addiction, nicotine addiction or drug addiction.

109. A method according to claim 108,
wherein said drug addiction is addiction to opioids.

110. A method for improving organ and cell survival, comprising the step of:
administering to a patient in need of such treatment an effective amount of a compound according to claim 1.

111. A method for providing cardioprotection following myocardial infarction, comprising the step of:
administering to a patient in need of such treatment an effective amount of a compound according to claim 1.

112. A method for reducing the need for anesthesia, comprising the step of:
administering to a patient in need of such treatment an effective amount of a compound according to claim 1.

112. A method of producing or maintaining an anesthetic state, comprising the step of:
administering to a patient in need of such treatment an effective amount of a compound according to claim 1.

114. A method according to claim 113, further comprising the step of:
administering to said patient an anesthetic agent selected from the group consisting of an inhaled anesthetic, a hypnotic, an anxiolytic, a neuromuscular blocker and an opioid.

115. A method according to claim 114,
wherein said compound and said anesthetic agent are co-administered.

116. A compound according to claim 1 which is radio-labeled.

117. A compound according to claim 1 which is isotopically-labeled.
